(19) United States
(12) Patent Application Publication
(10) Pub. No.: US 2026/0182980 A1
(43) Pub. Date: Jul. 2, 2026

(54) Knotless Orthopedic Stabilization System and Related Methods

(71) Applicant: Dunamis Medical Technologies, LLC, Greenville, AL (US)

(72) Inventors: Prithviraj Chavan, Greenville, AL (US); Forrest Samuel, Carlsbad, CA (US)

(21) Appl. No.: 19/131,415

(22) PCT Filed: Nov. 20, 2023
(86) PCT No.: PCT/US2023/080590

§ 371 Date: May 20, 2025

Related U.S. Application Data
(60) Provisional application No. 63/426,778, filed on Nov. 20, 2022.
(60) Provisional application No. 63/591,010, filed on Oct. 17, 2023.

(51) Int. Cl. A61B 17/04; A61B 17/84; A61F 2/08

(52) U.S. Cl. CPC A61B 17/0401; A61F 2/0811; A61B 2017/0404; A61B 2017/0456; A61B 2017/0464; A61B 17/846; A61F 2002/0852; A61F 2002/0882

(57) ABSTRACT

A knotless orthopedic fixation system comprising a surgical button, locking element, and flexible fixation member configured for use in bone-to-bone and soft tissue-to-bone repair, by knotlessly securing the flexible fixation member under tension. The button may be engaged with a first bone segment, and the flexible fixation member is secured to a second bone segment, soft tissue portion, or other member (e.g., plate, secondary button, etc.) and then passed through the button, to which it is locked under tension by way of the locking element. When assembled with the button and locking member, the flexible fixation member comprises a distal looped portion (e.g., attached to tissue, bone, or other member), one or more proximal looped portions (e.g., passed through transverse openings of the elongated flange), and free ends that extend proximally from the button after passing through the “pinch points”, which are pulled to tension the system.

### CROSS-REFERENCES TO RELATED APPLICATIONS

The present application is national stage entry under 35 U.S.C. § 371 of PCT application No. PCT/US23/80590, filed Nov. 20, 2023, which claims the benefit of priority from U.S. Provisional Patent Application Ser. No. 63/426,778 filed on Nov. 20, 2022, and U.S. Provisional Patent Application Ser. No. 63/591,010 filed on Oct. 17, 2023, the complete disclosures of which are hereby expressly incorporated by reference into this disclosure as if set forth fully herein.

### FIELD

The present disclosure relates generally to soft tissue repair fixation, and more specifically to an improved knotless assembly and method of fixation for securing soft tissue, bone, ligaments, tendons, grafts, allografts, and tension members.

### BACKGROUND

Tendon, ligament, and joint capsular injuries account for 45 percent of orthopedic injuries who would seek medical attention. Tendon injuries alone account for 30 million people annually which result in enormous amount of financial and physical burden to both the individual and the economy. Most devices used for repair of these injuries require knot-tying or knotless fixation for securing soft tissue to bone or bone to bone.

Traditionally, devices that use a button system for soft tissue to bone or bone to bone repair have several challenges that can impact surgical outcomes. These challenges include the need to tie a knot, using secondary fixation (i.e., screws) wiper windshield effect resulting in bone loss, cystic changes, device failures, the sutures used from fixation can fail by friction between the suture and the bone or an implant.

The knotless orthopedic stabilization system disclosed herein prevents these clinical failures or poor surgical outcomes. The system of the present disclosure has several advantages over the commercially available devices in the marketplace. The device and method of repair described herein utilizes a button device along with fixation members that can assist with performing this repair. The device disclosed herein may be used for various soft tissue to bone and bone to bone applications. The device disclosed herein may also be used as an adjunct for fracture fixation. Some of the examples of applications include syndesmosis repair, AC repair, ACL repair, PCL repair, Fibula fractures, etc.

### SUMMARY

In some embodiments, the knotless stabilization system disclosed herein includes a base member (or “button”), a locking element, and a flexible fixation member. In some embodiments, the base member and locking element cooperate to lock the fixation member (e.g., surgical suture, surgical tape, tensioning member, etc.) therebetween after a desired tension has been applied to the fixation member. By way of example, the base member may have any suitable shape, including but not limited to circular, oval, oblong, rectangular, etc. By way of example, the base member may have one or more openings configured to receive a locking element therethrough. By way of example only, the base member may comprise a single or multi-hole button, plate, surgical nail (e.g., fibular nail), and the like. In some embodiments, the base member comprises an orthopedic button having an oblong shape with a longitudinal axis and a transverse axis.

In some embodiments, the base member includes a top or proximal surface, a bottom or distal surface, a central recess formed within the top surface and including a recessed surface, and a central aperture positioned within the central recess and extending between the recessed surface and bottom surface. In some embodiments, the button further includes a sloped circumferential mating surface surrounding the central recess having a slope extending into the central recess from the top surface. In some embodiments, the sloped mating surface has a generally linear slope. In some embodiments, the sloped mating surface may have a generally concave slope. In some embodiments, the sloped mating surface may have a generally convex slope. In some embodiments, the sloped mating surface may have a slope angle in the range of 1° to 90°. By way of example, the sloped mating surface is complementary to the sloped mating surface of the locking element and cooperates with the sloped mating surface of the locking element to pinch the fixation element therebetween under tension (e.g., at “pinch points”), to knotlessly lock the button assembly during use.

In some embodiments, the central aperture may have a first pair of opposing shaped cutaway portions each dimensioned to receive a portion of the elongated flange of the locking element therein when the locking element is mated with the button. By way of example, the shaped cutaway portions have a shape that is complementary to the cross-sectional shape of the flange so that the cutaway portions prevent rotational movement of the locking element during tightening of the fixation member. In some embodiments, the central aperture may further include a second pair of opposing shaped cutaway portions positioned around the circumference of the central aperture from the first pair of shaped cutaway portions. By way of example only, the second pair of shaped cutaway portions are configured to enable passage of the fixation element (e.g., suture, tensioning member, etc.) through the button during fixation and helps to limit circumferential migration of the fixation element around the central aperture during use. Thus, in some embodiments, the “pinch points” where tension locking of the fixation member occurs between respective mating surfaces of the button and locking element are located proximate the second shaped cutaway portions.

In some embodiments, the first pair of shaped cutaway portions are positioned at an oblique angle relative to the longitudinal axis and the transverse axis. In some embodiments, the button may have more than one pair of first shaped opposing cutaway portions, for example for larger buttons or fixation plates that may accommodate multiple locking elements.

In some embodiments, the second pair of shaped cutaway portions are positioned at an oblique angle relative to the longitudinal axis and/or the transverse axis. In some embodiments, the button may have more than one pair of second shaped opposing cutaway portions, for example for larger buttons or fixation plates that may can accommodate multiple locking elements, or for instances in which multiple fixation members are used.

In some embodiments, the locking element has a head and elongated flange extending distally from the head. By way of example, the head may have any shape, including but not limited to circular, square, oblong, oval, rectangular, triangular, etc. In some embodiments, the head has a top or proximal side, a bottom or distal side, and a sloped mating surface provided on the bottom side extending around the periphery of the head. By way of example, the sloped mating surface is complementary to the mating surface of the button and cooperates with the sloped mating surface of the button to pinch the fixation element therebetween under tension (e.g., at “pinch points”), to knotlessly lock the button assembly during use. In some embodiments, the sloped mating surface has a generally linear slope. In some embodiments, the sloped mating surface may have a generally convex slope. In some embodiments, the sloped mating surface may have a generally concave slope. In some embodiments, the head may include a pair of concave recesses similar to concave recesses configured for passage of the fixation member therethrough.

In some embodiments, the elongated flange may have two or more transverse openings spaced vertically apart from one another. In some embodiments, the elongated flange includes a first transverse opening configured to allow passage of one or more loops of the fixation member therethrough. In some embodiments, the elongated flange may include a second transverse opening configured to allow passage of a different one or more loops of the fixation member therethrough. In some embodiments, passing only one loop of the fixation member through each transverse opening may make tensioning of the fixation member easier in that it reduces friction that may occur when multiple strands of the fixation member pass through the same transverse opening. In some embodiments, the outer edges of the transverse openings may be smooth, curved, and/or rounded to reduce friction on the fixation member as it passes through the transverse openings. By way of example only, the elongated flange may have rounded lateral sides having a complementary size and shape corresponding to the first pair of shaped cutaway portions of the button. By way of example, the elongated flange has a proximal end and a distal end. The proximal end is the portion of the elongated flange that interfaces with and extends from the head of the locking element. In some embodiments, the head has a peripheral cutaway portion positioned between the bottom side of the head and proximal end of the elongated flange. In some embodiments, the distal end may comprise a smooth and/or rounded surface to minimize trauma to nearby patient bone or tissue.

In some embodiments, the elongated flange may have a maximum width dimension having a value of not more than 2.5 mm. In some embodiments, the elongated flange may have a maximum width dimension having a value between 1 mm and 2.5 mm. In some embodiments, the elongated flange may have a length dimension measured from the proximal end to the distal end. In some embodiments, the length dimension has a value between 1 mm and 30 mm. In some embodiments, the elongated flange may have a length dimension having a value of 4 mm. In some embodiments, the elongated flange (as well as the base member and locking element) may be larger to fit specific anatomy.

By way of example, the knotless orthopedic fixation system is configured for use in bone-to-bone and soft tissue-to-bone repair, by knotlessly securing the flexible fixation member under tension. In some embodiments, the button is engaged with a first bone segment, and the flexible fixation member is secured to a second bone segment, soft tissue portion, or other member (e.g., plate, secondary button, etc.) and then passed through the button, to which it is locked under tension by way of the locking element. By way of example, when assembled with the button and locking member, the flexible fixation member comprises a distal looped portion (e.g., which is the portion of the fixation member that is attached to tissue, bone, or other member), one or more proximal looped portions (e.g., which are passed through transverse openings of the elongated flange), and free ends that extend proximally from the button after passing through the “pinch points”.

By way of example, tensioning of the fixation members is performed by pulling the free ends of the fixation members which will reduce the distance between the proximal looped end(s) and the distal looped end(s), and is completed when appropriate tension is achieved between two firm end points that increase the tension in the fixation member which allows for pinching of the fixation members at pinch points between the base member and locking element as described above, thereby maintaining the stabilization system in a locked state.

In some embodiments, the knotless orthopedic fixation system may include a retainer mechanism configured to keep the system in a locked state even if the tension applied to the fixation member(s) is insufficient to maintain the locking element in a locked state on its own. In some embodiments, the retainer mechanism may comprise one or more lateral flanges or other physical structure provided on the elongated flange of the locking element that are configured to interact with one or more receiving elements provided on the button to retain the locking element in a locked state. In some embodiments, the retainer mechanism may comprise one or more flanges or other physical structure provided on the button that interacts with a receiving element on the elongated flange of the locking element.

In some embodiments, the button may have at least one recess or undercut formed in the bottom surface that intersect with at least one of the first pair of shaped cutaway portions. By way of example, the recess has a contact surface configured to interface with the trailing surface of the lateral flange. In some embodiments, the contact surface is planar. In some embodiments, the contact surface may be curved. In some embodiments, the lateral flange is configured to interact with the recess to lock the locking element to the button.

Once the locking element has been pushed or pulled through the central aperture so that the apexes of the lateral flanges are seated within the recesses, the locking element is blocked from proximal movement (which may cause unwanted loosening of the tension in the fixation element) by virtue of a physical interaction between the trailing surface of the lateral flange and the planar surface of the recess.

In some embodiments, the base member comprises a round orthopedic button. In some embodiments, the base member includes a top or proximal surface, a bottom or distal surface, a central recess formed within the top surface, an elongated central aperture positioned within the central recess, and a pair of lateral apertures positioned on either side of the central aperture. By way of example, the central aperture is elongated and has a shape complementary to the shape of the elongated flange of the locking element to prevent the locking element from rotating when mated with the base member. The lateral apertures are configured to enable passage of one or more fixation members therethrough. In a preferred embodiment, each lateral aperture is configured to receive a free end of the fixation member therethrough. In some embodiments, the button further includes a sloped circumferential mating surface surrounding the central recess having a slope extending into the central recess from the top surface. By way of example, the sloped mating surface is complementary to the sloped mating surface of the locking element and cooperates with the sloped mating surface of the locking element to pinch the fixation element therebetween under tension (e.g., at “pinch points”), to knotlessly lock the button assembly during use.

In some embodiments, the elongated flange may have a single transverse opening positioned near the distal end and configured to allow passage of one or more proximal loops of the fixation member therethrough. In some embodiments, the transverse opening has a width or diameter dimension. In some embodiments, the transverse opening is spaced from the proximal end a distance that is more than double the width dimension of the transverse opening. In some embodiments, the transverse opening is spaced from the proximal end a distance that is more than triple the width dimension of the transverse opening.

In some embodiments, the knotless orthopedic stabilization system may be used in an orthopedic fixation procedure with a plate, rod, nail, and the like as a supplemental stabilizing element rather than a primary fixation element. In some embodiments, the system may be used with a fibular nail to stabilize the fixation procedure. For example, a transverse bone tunnel may be drilled through a fibula and also the tibia at a location corresponding to a hole in the fibular nail. A supplemental button or plate is then shuttled through the bone tunnel to the far side of the tibia where it is positioned and secured with one or more fixation members (e.g., by engaging with distal looped ends of the fixation member). Proximal looped ends of the fixation member engage the transverse opening of the elongated flange, which is positioned through the hole in the fibular nail. In some embodiments, the elongated flange may extend at least partially into the bone tunnel in the fibula and/or tibia. The construct is then tensioned by pulling on the free ends of the fixation member as described herein.

In some embodiments, the base member comprises a round orthopedic button having a stabilizer member or jacket extending distally therefrom to provide stability of the fixation construct when used as a supplemental fixation element (e.g., with a plate, nail, etc.) or as a primary fixation element (e.g., when inserted into a bone tunnel). By way of example, the stabilizing jacket is configured for insertion or through into a bone channel or another fixation member (e.g., plate, nail, etc.) and stabilizes the elongated flange of the locking element to prevent the elongated flange from moving during use (e.g., preventing back and forth rocking or “windshield wiper” motion) to ensure a more stable fixation construct.

In some embodiments, the knotless orthopedic stabilization system may be modified for use with a fixation plate. In some embodiments, the bottom side of the base member includes a plurality of boss members that are configured for insertion into an existing fixation aperture or screw hole of a fixation plate, for example, so that the system may then be used with the plate in the manner described consistently herein with other embodiments. In some embodiments, the height of the boss members can be varied to alter the orientation of the locking element relative to the plate, for example to change the angle of the elongated flange as it extends through the plate, which may enable fixation with the flexible fixation member at a more extreme angle.

In some embodiments, the locking element may include more than two transverse openings formed therein to enable multiple fixation members to be used and still have a single strand per transverse aperture. In some embodiments, the fixation members may be attached at a distal looped end to a multiple of tissue segments, bone pieces, or other members.

In some embodiments, the knotless fixation assembly disclosed herein may also be locked into a fixation plate in a certain direction to allow for maximum structural support by dialing or biasing the passage of the fixation members at an angle that may be performed by the operator of the device. In some embodiments, the locking element may be pivotable so that the flange may extend at different or variable angles through the button and then be locked in place. In some embodiments, the locking element and/or button and/or fixation plate may be modified to allow for various locking angles. In some embodiments, a fixation plate or button may include an aperture configured to receive the button therein having an angled slot configured to receive the flange of the locking element threrethrough. By way of example, the slot may be angled or directionally oriented in the direction that the user wants the flange to extend. In some embodiments, the same feature can be achieved by way of an extender on the button (for example).

In any embodiment described herein, tensioning of the fixation members is performed by pulling the free ends of the fixation members which will reduce the distance between the proximal looped end(s) and the distal looped end(s), and is completed when appropriate tension is achieved between two firm end points that increase the tension in the fixation member which allows for pinching of the fixation members at pinch points between the base member (and variants) and locking element (and variants) as described herein, thereby maintaining the knotless stabilization system in a locked state.

Other embodiments may include single or multiple openings of the locking element, which by way of example only may be 4 mm or more in length. For example, in some embodiments, the flange portion of the locking element may be between 1 mm and 30 mm in length. In some embodiments, the button may have one or more openings. In some embodiments, the button may have extensions that extend into bone and provide additional stability to the bone, device, or other implants used. In some embodiments, the locking assembly has more than one mating surface and can be used for fixation members for soft tissue, and/or bone to bone, and/or the loops may be connected to another device on the far cortex (for example).

In some embodiments, the locking button assembly with fixation members that connect to one or multiple fixation members that allow for tensioning a plurality of constructs in conjunction with one or more knotless assemblies.

In some embodiments, the button and/or locking element can be made from titanium, stainless steel, peek, polymers, 3D-printing, or similar materials used in the industry to manufacture the button device. The manufacturing process can be machining, molding, 3D-printing, or other similar acceptable processes used in the industry. The fixation members can be suture, sutures, nitinol, other materials that can be used to fix soft tissue to bone fixation members may be braided, non-braided, can have a core or coreless.

In some embodiments, the knotless orthopedic stabilization system comprises a button and a locking element extending through an aperture in the button and into bone, for example. By way of example only, the locking element may prevent friction between the bone, implant, and fixation members (e.g., sutures) and/or devices that function as fixation members.

In some embodiments, the locking element includes a head portion and a flange extending from the head portion. By way of example, the head portion engages with the button, and the flange extends through an aperture in the button. In some embodiments, the flange may have one or more openings which can be used to pass flexible fixation members (e.g., surgical sutures, tape, tension members, etc.). These openings can improve the experience by minimizing the friction or the resistance encountered by the surgeon while tensioning the device. These openings can also allow for multiple fixation members that can increase the strength of the repair and offer the option of connecting these fixation members to one or multiple devices on the opposite side of the repair.

In some embodiments, the button and locking element assembly can function as a bolt (for example) by extending at least partially through a device used for fracture fixation (e.g., a fibular nail) to secure the device to bone and thereby prevent migration of the fracture fixation device or damage to the fixation members. In some embodiments, the button and locking element can lock within the fracture fixation device to provide a bridging construct.

In some embodiments, the elongation of the button assembly (e.g., the locking element flange) positions the fixation members closer to the location of soft tissue tear or tissue damage to enable the fixation members to be as anatomical as possible to the site of soft tissue tear or tissue damage (e.g., syndesmosis repair).

In some embodiments, the fixation members may additionally have stabilization features that occupy an opening or pilot hole drilled into bone thereby preventing excessive motion between the device, the fixation members, and the bone. In some embodiments, the elongated flange of the locking element may be sized and configured to completely fill a bore formed in bone to prevent excessive motion between the device, fixation members, and the bone. For example, the bore or bone tunnel may be occupied by the locking pin or the button and/or extensions of these buttons that prevent excessive motion, (e.g., windshield wiper effect) that may lead to damage to bone and/or loosening of the repair.

In some embodiments, the locking element design that provides the ability to connect with multiple fixation points, these fixation points can be additional devices, grafts, or soft tissue.

In some embodiments, the locking element may have one or more extensions that act as a platform to provide stability to bone or nail construct.

As additional description to the embodiments described below, the present disclosure describes the following embodiments.

Embodiment 1 is an orthopedic stabilization system comprising: a base member having a proximal side, a distal side, a central recess formed in the proximal side, and a central opening positioned within the central recess and extending therethrough from the proximal side to the distal side; a locking element having a proximal side, a distal side, and an elongated flange having an oblong cross-sectional shape extending distally from the distal side of the locking element, the elongated flange having at least two transverse openings spaced longitudinally apart from one another, the locking element configured to mate with the base member such that the elongated flange extends through the central opening and the at least two transverse openings are positioned distal of the distal side of the base member; and a flexible fixation member configured to form into a looped orientation having two free ends, a distal loop portion, and a plurality of proximal loop portions, the flexible fixation member configured to pass through the central opening in the base member and between the base member and the locking element such that the two free ends are disposed outwardly in a proximal direction from the proximal side of the base member; wherein a first of the plurality of proximal loop portions is configured to pass through a first of the at least two transverse openings in the elongated flange, a second of the plurality of proximal loop portions is configured to pass through a second of the at least two transverse openings in the elongated flange, and the distal loop portion is configured to engage a tissue, bone, or other member.

Embodiment 2 is the orthopedic stabilization device of embodiments 1, wherein the central opening has a perimeter and further comprises a first pair of opposing shaped perimeter recesses formed within said perimeter, each of the first pair of opposing shaped perimeter recesses configured to flushly engage a portion of the elongated flange.

Embodiment 3 is the orthopedic stabilization device of embodiments 1 or 2, wherein the central opening further comprises a second pair of opposing shaped perimeter recesses formed within said perimeter and different from the first pair of opposing shaped perimeter recesses, the second pair of opposing shaped perimeter recesses configured to enable passage of the flexible fixation member through the base member.

Embodiment 4 is the orthopedic stabilization device of any of embodiments 1 through 3, wherein the first pair of opposing perimeter recesses has a first size dimension, the second pair of opposing perimeter recesses has a second size dimension, and the first size dimension is greater than the second size dimension.

Embodiment 5 is the orthopedic stabilization device of any of embodiments 1 through 4, wherein the first pair of opposing perimeter recesses and the second pair of opposing perimeter recesses are distributed about the perimeter of the central opening at 90° intervals from one another.

Embodiment 6 is the orthopedic stabilization device of any of embodiments 1 through 5, wherein the base member has a longitudinal axis extending therethrough, and the first pair of opposing shaped perimeter recesses are positioned about the perimeter of the central opening at an oblique angle relative to the longitudinal axis.

Embodiment 7 is the orthopedic stabilization device of any of embodiments 1 through 6, wherein the oblique angle is 45°.

Embodiment 8 is the orthopedic stabilization device of any of embodiments 1 through 7, wherein the locking element further comprises one or more retention members positioned on the elongated flange and configured to engage the base member to prevent dislodging of the locking element from the base member when the locking element is coupled to the base member and the retention members are positioned distal of the base member.

Embodiment 9 is the orthopedic stabilization device of any of embodiments 1 through 8, wherein the retention members are configured to deform as the elongated flange is inserted through the central opening during coupling of the base member and locking element.

Embodiment 10 is the orthopedic stabilization device of any of embodiments 1 through 9, wherein the retention members are configured to return to their normal configuration when the retention members are distally clear of the central opening during coupling of the base member and locking element.

Embodiment 11 is the orthopedic stabilization device of any of embodiments 1 through 10, wherein the base member has one or more undercut recesses formed in the distal side and configured to receive the retention members therein, when the retention members are distally clear of the central opening during coupling of the base member and locking element.

Embodiment 12 is the orthopedic stabilization device of any of embodiments 1 through 11, wherein central recess has a first peripheral mating surface.

Embodiment 13 is the orthopedic stabilization device of any of embodiments 1 through 12, wherein the distal side of the locking element as a second peripheral mating surface.

Embodiment 14 is the orthopedic stabilization device of any of embodiments 1 through 13, the flexible fixation member is configured to pass between the first and second peripheral mating surfaces.

Embodiment 15 is the orthopedic stabilization device of any of embodiments 1 through 14, wherein the first and second mating surfaces are configured to cooperate to capture the flexible fixation members with one or more pinch points that maintain the flexible fixation members under tension in a locked state.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a perspective view of an example of a knotless orthopedic stabilization system according to some embodiments;

FIG. 2 is a bottom perspective view of the stabilization system of FIG. 1, according to some embodiments;

FIG. 3 is a front perspective view of the stabilization system of FIG. 1, according to some embodiments;

FIG. 4 is a perspective view of a base member forming part of the stabilization system of FIG. 1, according to some embodiments;

FIG. 5 is a perspective view of a locking element forming part of the stabilization system of FIG. 1, according to some embodiments;

FIG. 6 is a side view of the locking element of FIG. 5, according to some embodiments;

FIG. 7 is a perspective view of another example of a knotless orthopedic stabilization system according to some embodiments;

FIGS. 8-11 are front perspective, bottom perspective, side plan, and bottom plan views, respectively, of the stabilization system of FIG. 7;

FIG. 12 is an exploded perspective view of the stabilization system of FIG. 7, according to some embodiments;

FIGS. 13-15 are perspective, top plan, and bottom plan views, respectively, of a base member forming part of the stabilization system of FIG. 7, according to some embodiments;

FIG. 16-17 are front plan and side plan views, respectively, of a locking element forming part of the stabilization system of FIG. 7, according to some embodiments;

FIG. 18 is a perspective view of another example of a knotless orthopedic stabilization system according to some embodiments;

FIGS. 19-20 are perspective and exploded perspective views, respectively, of the stabilization system of FIG. 18, according to some embodiments;

FIG. 21 is a perspective view of another example of a knotless orthopedic stabilization system according to some embodiments;

FIG. 22 illustrates the stabilization system of FIG. 21 in use with a femoral nail, according to some embodiments;

FIG. 23 is a perspective view of another example of a knotless orthopedic stabilization system according to some embodiments;

FIGS. 24-26 are perspective, front plan, and side plan views, respectively, of the stabilization system of FIG. 23, according to some embodiments;

FIGS. 27-30 are top perspective, bottom perspective, top plan, and bottom plan views, respectively, of a base member forming part of the stabilization system of FIG. 23, according to some embodiments;

FIGS. 31-32 are perspective and plan views, respectively, of a locking element forming part of the stabilization system of FIG. 23, according to some embodiments;

FIG. 33 is a perspective view of another example of a knotless orthopedic stabilization system according to some embodiments;

FIG. 34 is an exploded perspective view of the stabilization system of FIG. 33, according to some embodiments;

FIG. 35 is a bottom perspective view of another example of a knotless orthopedic stabilization system according to some embodiments;

FIGS. 36-37 are perspective and exploded views, respectively, of another example of a knotless orthopedic stabilization system according to some embodiments;

FIG. 38 is a plan view of another example of a knotless orthopedic stabilization system according to some embodiments;

FIG. 39 is a plan view of the stabilization system of FIG. 38 with secondary fixation buttons, according to some embodiments;

FIG. 40 is a top plan view of a plate including an angled slot configured to receive a knotless stabilization system of FIG. 18, according to some embodiments; and

FIG. 41 is a side plan view of the plate of FIG. 40 with a knotless stabilization system of FIG. 18, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers'specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The knotless orthopedic stabilization system and related methods disclosed herein boasts a variety of inventive features and components that warrant patent protection, both individually and in combination.

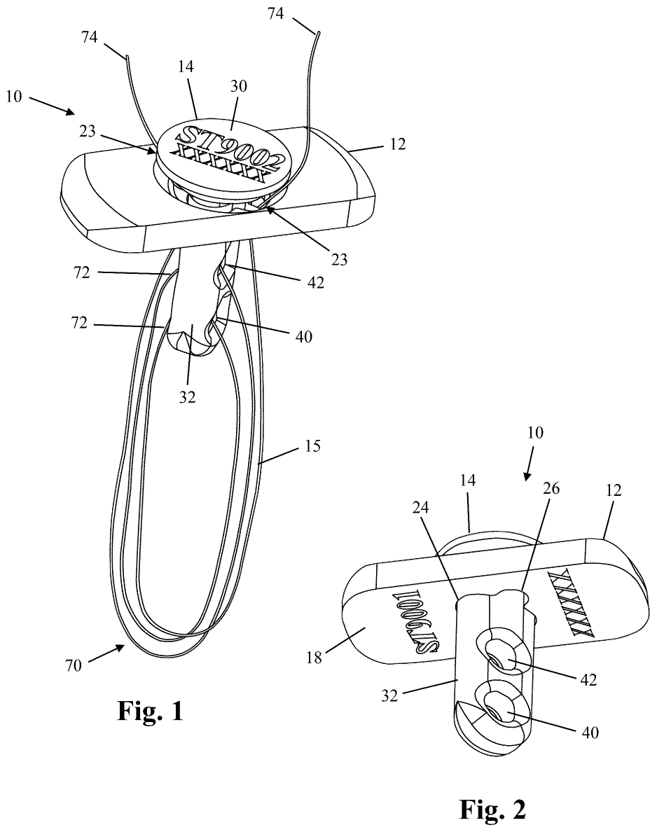

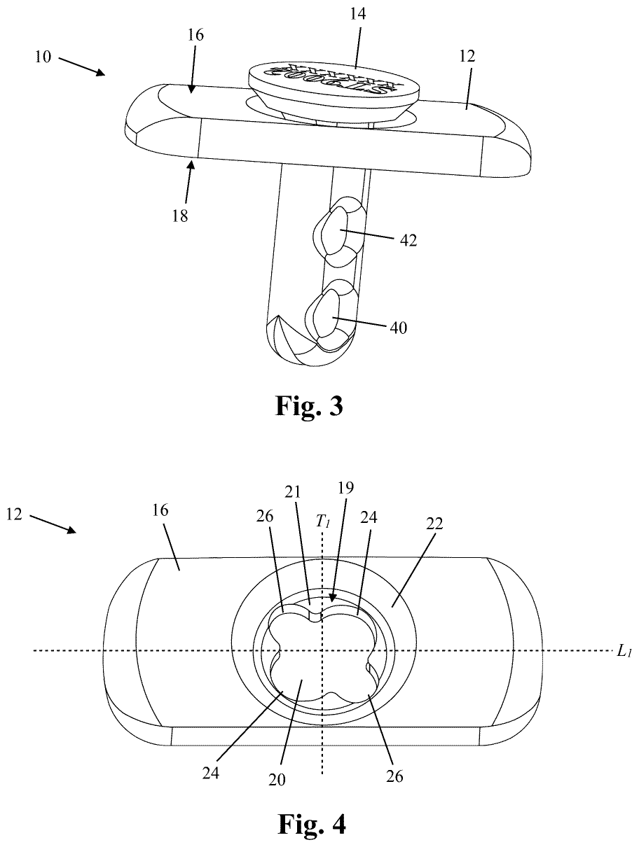

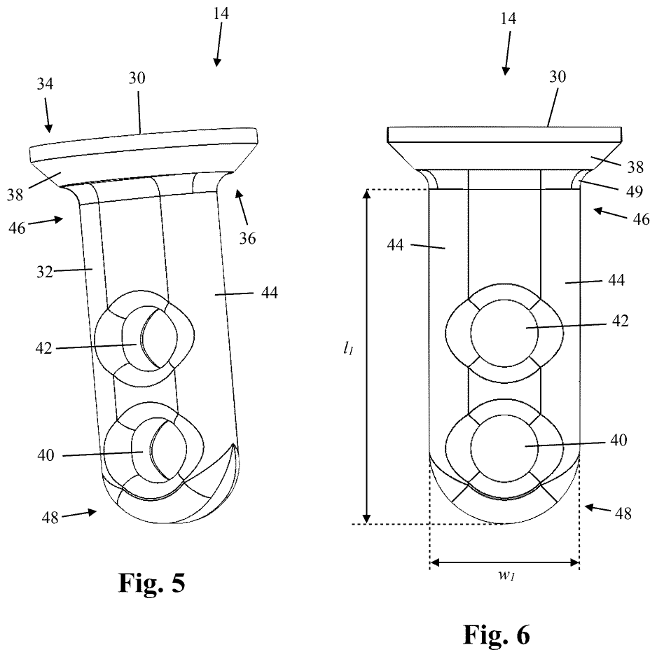

FIGS. 1-6 illustrate an example of a knotless orthopedic stabilization system 10 according to some embodiments of the present disclosure. By way of example, the knotless stabilization system 10 includes a base member (or “button”) 12, a locking element 14, and a flexible fixation member 15. In some embodiments, the base member 12 and locking element 14 cooperate to lock the fixation member 15 (e.g., surgical suture, surgical tape, tensioning member, etc.) therebetween after a desired tension has been applied to the fixation member. By way of example, the base member 12 may have any suitable shape, including but not limited to circular, oval, oblong, rectangular, etc. By way of example, the base member may have one or more openings configured to receive a locking element therethrough. By way of example only, the base member 12 may comprise a single or multi-hole button, plate, surgical nail (e.g., fibular nail), and the like. By way of example only, in the embodiment shown and described in FIGS. 1-6, the base member 12 comprises an orthopedic button having an oblong shape with a longitudinal axis L₁ and a transverse axis T₁.

In some embodiments, the base member 12 includes a top or proximal surface 16, a bottom or distal surface 18, a central recess 19 formed within the top surface 16 and including a recessed surface 21, and a central aperture 20 positioned within the central recess 19 and extending between the recessed surface 21 and bottom surface 18. In some embodiments, the button 12 further includes a sloped circumferential mating surface 22 surrounding the central recess 19 having a slope extending into the central recess 19 from the top surface 16. In some embodiments, the sloped mating surface 22 has a generally linear slope. In some embodiments, the sloped mating surface 22 may have a generally concave slope. In some embodiments, the sloped mating surface 22 may have a generally convex slope. In some embodiments, the sloped mating surface 22 may have a slope angle in the range of 1° to 90°. By way of example, the sloped mating surface 22 is complementary to the sloped mating surface 38 of the locking element 14 and cooperates with the sloped mating surface 38 of the locking element 14 to pinch the fixation element 15 therebetween under tension (e.g., at “pinch points” 23), to knotlessly lock the button assembly 10 during use.

In some embodiments, the central aperture 20 may have a first pair of opposing shaped cutaway portions 24 each dimensioned to receive a portion of the elongated flange 32 of the locking element 14 therein when the locking element 14 is mated with the button 12. By way of example, the shaped cutaway portions 24 have a shape that is complementary to the cross-sectional shape of the flange 32 so that the cutaway portions 24 prevent rotational movement of the locking element 12 during tightening of the fixation member. In some embodiments, the central aperture 20 may further include a second pair of opposing shaped cutaway portions 26 positioned within a range of 60-90° around the circumference of the central aperture 20 from the first pair of shaped cutaway portions 26. In a preferred embodiment, the first and second pairs of shaped cutaway portions 24, 26 are spaced evenly apart from one another at 90° intervals around the circumference of the central aperture 20. By way of example only, the second pair of shaped cutaway portions 26 are configured to enable passage of the fixation element 15 (e.g., suture, tensioning member, etc.) through the button 12 during fixation and helps to limit circumferential migration of the fixation element 15 around the central aperture 20 during use. Thus, in some embodiments, the “pinch points” 23 where tension locking of the fixation member 15 occurs between respective mating surfaces 22, 38 of the button 12 and locking element 14 are located proximate the second shaped cutaway portions 26. In some embodiments, the first pair of opposing shaped cutaway portions 24 may have the same size and shape as the second pair of opposing shaped cutaway portions 26 such that the elongated flange 32 of the locking element 14 may be coupled with the button 12 in any orientation. In some embodiments, the first pair of opposing shaped cutaway portions 24 may have a different size and shape from the second pair of opposing shaped cutaway portions 26 such that the elongated flange 32 of the locking element 14 may be coupled with the button 12 in only one orientation.

In some embodiments, the first pair of shaped cutaway portions 24 are positioned at an oblique angle relative to the longitudinal axis L₁ and the transverse axis T₁. In some embodiments, the first pair of shaped cutaway portions 24 are offset by 45° relative to the longitudinal axis L₁ and/or the transverse axis T₁. In some embodiments, the offset degree of the first pair of shaped cutaway portions 24 may be between 30° and 60° relative to the longitudinal axis L₁ and/or the transverse axis T₁. In some embodiments, the button 12 may have more than one pair of first shaped opposing cutaway portions 24, for example for larger buttons 12 or fixation plates that may accommodate multiple locking elements 14.

In some embodiments, the second pair of shaped cutaway portions 26 are positioned at an oblique angle relative to the longitudinal axis L₁ and/or the transverse axis T₁. In some embodiments, the second pair of shaped cutaway portions 26 are offset by 45° relative to the longitudinal axis L₁ and/or the transverse axis T₁. In some embodiments, the offset angle of the second pair of shaped cutaway portions 26 may be between 30° and 60° relative to the longitudinal axis L₁ and/or the transverse axis T₁. In some embodiments, the button 12 may have more than one pair of second shaped opposing cutaway portions 26, for example for larger buttons 12 or fixation plates that may can accommodate multiple locking elements 14, or for instances in which multiple fixation members 15 are used.

By way of example, the oblique orientation of the first and second shaped cutaway portions 24, 26 of the base member 12 enable the base member 12 and locking element 14 to be manufactured in a smaller size than would be possible if the locking element 14 were oriented such that a transverse axis of the elongated flange 32 was positioned coaxial with either the longitudinal axis L₁ or transverse axis T₁ of the base member 12.

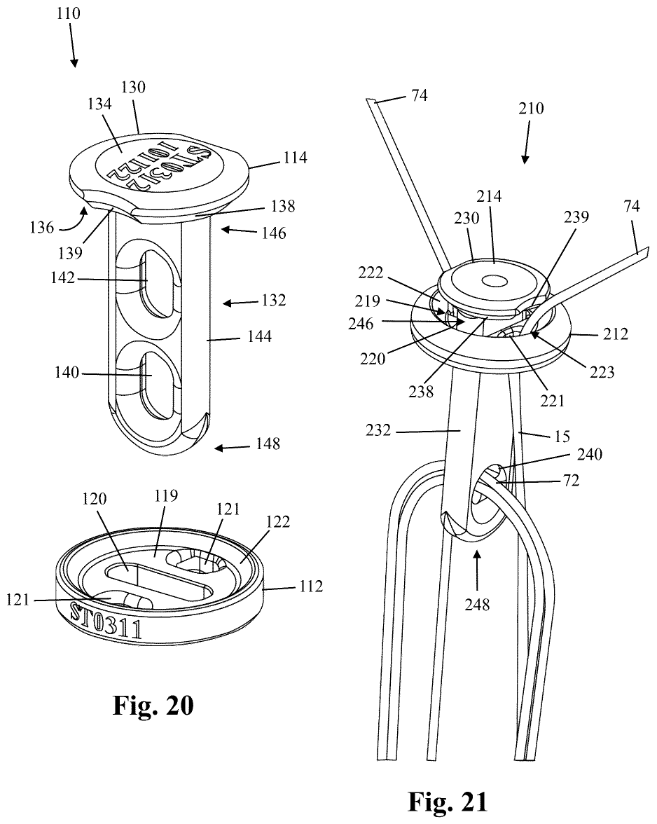

In some embodiments, the locking element 14 has a head 30 and elongated flange 32 extending distally from the head 30. By way of example, the head 30 may have any shape, including but not limited to circular (as shown by way of example only in FIG. 1), square, oblong, oval, rectangular, triangular, etc. In some embodiments, the head 30 has a top or proximal side 34, a bottom or distal side 36, and a sloped mating surface 38 provided on the bottom side 36 extending around the periphery of the head 30. By way of example, the sloped mating surface 38 is complementary to the mating surface 22 of the button 12 and cooperates with the sloped mating surface 22 of the button 12 to pinch the fixation element 15 therebetween under tension (e.g., at “pinch points” 23), to knotlessly lock the button assembly 10 during use. In some embodiments, the sloped mating surface 38 has a generally linear slope. In some embodiments, the sloped mating surface 38 may have a generally convex slope. In some embodiments, the sloped mating surface 38 may have a generally concave slope. In some embodiments, the head 30 may include a pair of concave recesses similar to concave recesses 139 shown by way of example in FIG. 20 configured for passage of the fixation member 15 therethrough.

In some embodiments, the elongated flange 32 may have two or more transverse openings spaced vertically apart from one another. In some embodiments, the elongated flange 32 includes a first transverse opening 40 configured to allow passage of one or more loops of the fixation member 15 therethrough. In some embodiments, the elongated flange 32 may include a second transverse opening 42 configured to allow passage of a different one or more loops of the fixation member 15 therethrough. In some embodiments, passing only one loop of the fixation member 15 through each transverse opening may make tensioning of the fixation member 15 easier in that it reduces friction that may occur when multiple strands of the fixation member 15 pass through the same transverse opening. In some embodiments, the outer edges of the transverse openings 40 and/or 42 may be smooth, curved, and/or rounded to reduce friction on the fixation member 15 as it passes through the transverse openings. By way of example only, the elongated flange 32 may have rounded lateral sides 44 having a complementary size and shape corresponding to the first pair of shaped cutaway portions 24 of the button 12. By way of example, the elongated flange 32 has a proximal end 46 and a distal end 48. The proximal end 46 is the portion of the elongated flange 32 that interfaces with and extends from the head 30 of the locking element 14. In some embodiments, the head 30 has a peripheral cutaway portion 49 positioned between the bottom side 36 of the head and proximal end 46 of the elongated flange 32. In some embodiments, the distal end 48 may comprise a smooth and/or rounded surface to minimize trauma to nearby patient bone or tissue.

In some embodiments, the elongated flange 32 may have a maximum width dimension w₁ having a value of not more than 2.5 mm. In some embodiments, the elongated flange 32 may have a maximum width dimension w₁ having a value between 1 mm and 2.5 mm. In some embodiments, the elongated flange 32 may have a length dimension l₁ measured from the proximal end 46 to the distal end 48. In some embodiments, the length dimension l₁ has a value between 1 mm and 30 mm. In some embodiments, the elongated flange 32 may have a length dimension l₁ having a value of 4 mm. In some embodiments, the elongated flange 32 (as well as the base member 12 and locking element 14) may be larger to fit specific anatomy.

Referring again to FIG. 1, the knotless orthopedic fixation system 10 is configured for use in bone-to-bone and soft tissue-to-bone repair, by knotlessly securing the flexible fixation member 15 under tension. In some embodiments, the button 12 is engaged with a first bone segment, and the flexible fixation member 15 is secured to a second bone segment, soft tissue portion, or other member (e.g., plate, secondary button, etc.) and then passed through the button 12, to which it is locked under tension by way of the locking element 14. By way of example, when assembled with the button 12 and locking member 14, the flexible fixation member 15 comprises a distal looped portion 70 (e.g., which is the portion of the fixation member 15 that is attached to tissue, bone, or other member), one or more proximal looped portions 72 (e.g., which are passed through transverse openings 40, 42 of the elongated flange 32), and free ends 74 that extend proximally from the button 12 after passing through the “pinch points” 23.

By way of example, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 23 between the base member 12 and locking element 14 as described above, thereby maintaining the stabilization system 10 in a locked state.

In some embodiments, the knotless orthopedic fixation system 10 may include a retainer mechanism configured to keep the system 10 in a locked state even if the tension applied to the fixation member(s) 15 is insufficient to maintain the locking element 14 in a locked state on its own. In some embodiments, the retainer mechanism may comprise one or more lateral flanges or other physical structure provided on the elongated flange 32 of the locking element 14 that are configured to interact with one or more receiving elements provided on the button 12 to retain the locking element 14 in a locked state. In some embodiments, the retainer mechanism may comprise one or more flanges or other physical structure provided on the button 12 that interacts with a receiving element on the elongated flange 32 of the locking element 14.

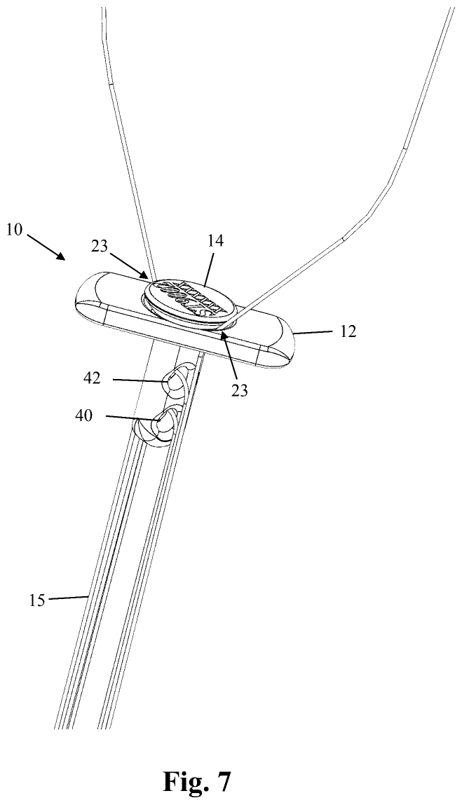

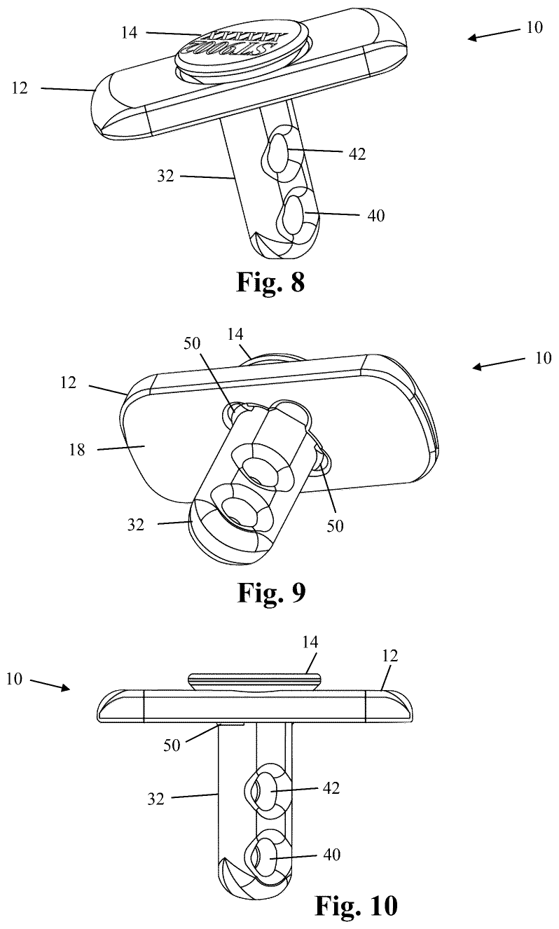

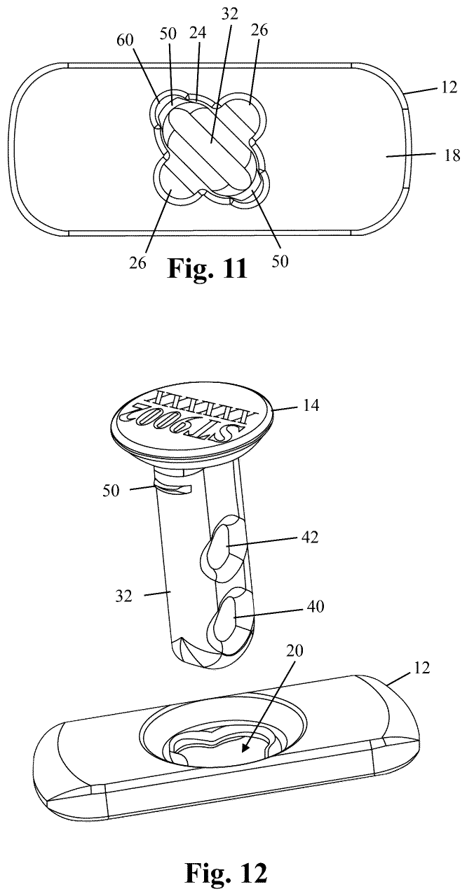

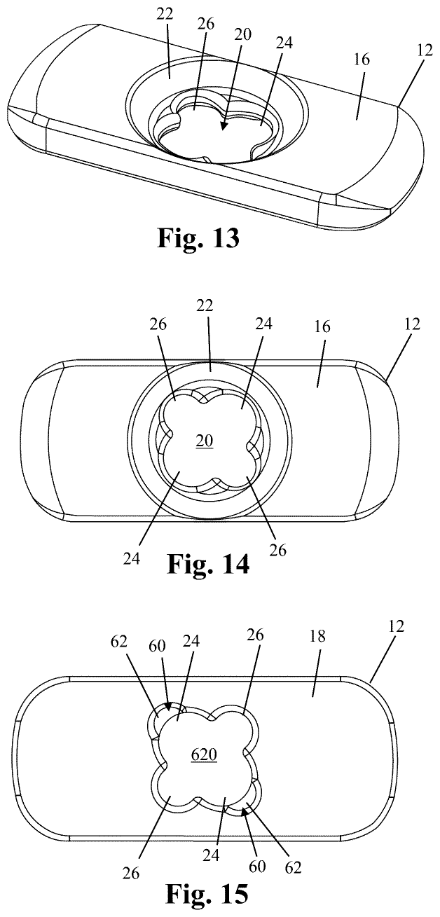

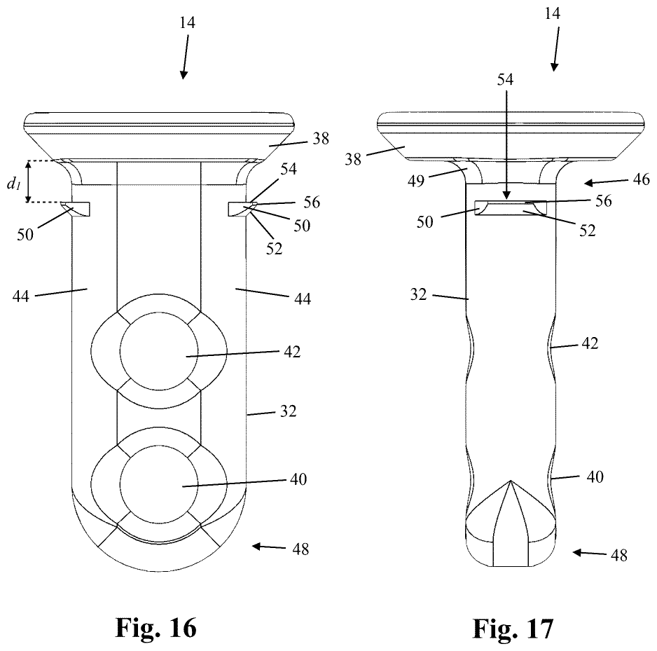

For example, in the embodiment shown and described in FIGS. 7-17, the retainer mechanism comprises one or more lateral flanges 50 provided on the elongated flange 32 of the locking element 14 that are configured to interact with one or more receiving elements provided on the button 12 to retain the locking element 14 in a locked state. In some embodiments, at least one lateral flange 50 is positioned on a rounded lateral side 44 of the locking element 14 at a distance d₁ from the bottom (or distal-most end) of the mating surface 38. In some embodiments, the locking element 14 may have a least two lateral flanges 50 with one lateral flange 50 positioned on each rounded lateral side 44 of the locking element 14, for example as shown in FIGS. 16-17. By way of example, each lateral flange 50 may have a leading or distal facing surface 52 and a trailing or proximal facing surface 54, which meet at an apex 56. In some embodiments, the distance between the apex 56 of a first lateral flange 50 and the apex of a second lateral flange 50 positioned on the opposite side of the locking element 12 defines a maximum width component of the elongated flange 32. In some embodiments, the leading surface 52 may be angled. In some embodiments, the trailing surface 54 may be planar.

In some embodiments, the button 12 may have at least one recess or undercut 60 formed in the bottom surface 18 that intersect with at least one of the first pair of shaped cutaway portions 24. By way of example, the recess 60 has a contact surface 62 configured to interface with the trailing surface 54 of the lateral flange 50. In some embodiments, the contact surface 62 is planar. In some embodiments, the contact surface 62 may be curved. In some embodiments, the lateral flange 50 is configured to interact with the recess 60 to lock the locking element 14 to the button 12.

By way of example, as the locking element 14 is advanced or pulled through the central aperture 20, the lateral flanges 50 advance past the outer rim of the first shaped cutaway portions 24 with slight deformation to make the clearance until the apex 56 of each lateral flange 50 enters the recess 60, at which point the lateral flanges 50 snap back to their original configurations, which may provide tactile and/or audible feedback to the user that the locking element 14 is secured to the button 12. In some embodiments, the locking element 14 may be advanced through the central aperture 20 by an application of a distally directed force by the user directly on the locking element 14, either manually (e.g., with a finger) or through use of an instrument configured to apply such force to the locking element 14. In some embodiments, the locking element 14 is advanced through the central aperture 20 during the process of tensioning the fixation elements, as applying tension to the fixation elements will cinch the construct by pulling the locking element 14 distally through the central aperture 20 of the button 12. In some embodiments, advancement of the lateral flanges 50 through the central aperture 20 occurs during the normal tensioning activity (in other words, with no additional force needed to engage the lateral flanges 50 with the recess 60). In some embodiments, both methods of advancing the locking element 14 through the central aperture 20 may be used.

In some embodiments, the distance d₁ between the lateral flange 50 (or more specifically, the trailing surface 54 of the lateral flange 50) and the bottom of the mating surface 38 is approximately equal to the thickness of the button 12 within the central recess 19 as measured from the recessed surface 21 to the contact surface 62 of the recess 60. In some embodiments, the distance d₁ between the lateral flange 50 (or more specifically, the trailing surface 54 of the lateral flange 50) and the bottom of the mating surface 38 is approximately equal to the thickness of the button 12 within the central recess 19 as measured from the bottom (e.g., distal-most) edge of the mating surface 22 of the central recess 19.

Once the locking element 14 has been pushed or pulled through the central aperture 20 so that the apexes 56 of the lateral flanges 50 are seated within the recesses 60, the locking element 14 is blocked from proximal movement (which may cause unwanted loosening of the tension in the fixation element) by virtue of a physical interaction between the trailing surface 54 of the lateral flange 50 and the planar surface of the recess 60.

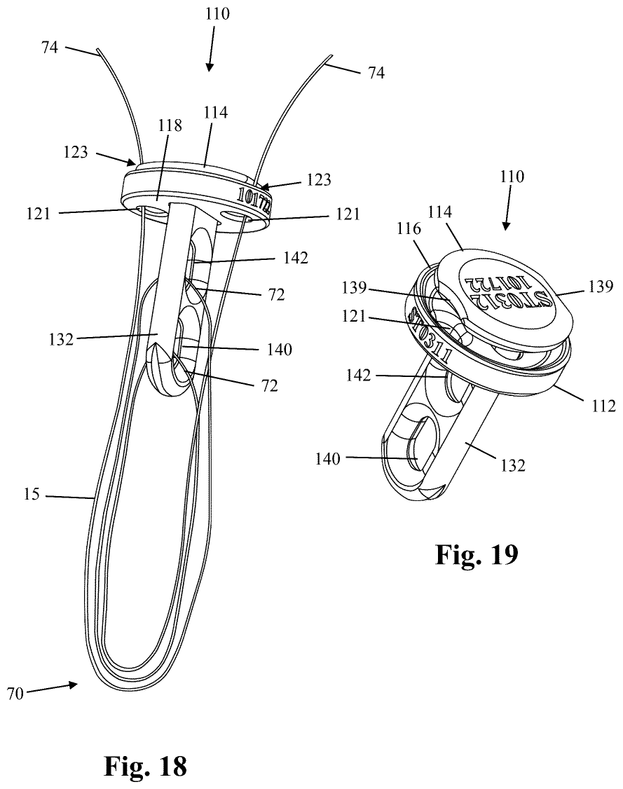

FIGS. 18-20 illustrate an example of a knotless orthopedic stabilization system 110 according to some embodiments of the present disclosure. By way of example, the knotless stabilization system 110 includes a base member (or “button”) 112, a locking element 114, and a flexible fixation member 15. In some embodiments, the base member 112 and locking element 114 cooperate to lock the fixation member 15 (e.g., surgical suture, surgical tape, tensioning member, etc.) therebetween after a desired tension has been applied to the fixation member. By way of example, the base member 112 may have any suitable shape, including but not limited to circular, oval, oblong, rectangular, etc. By way of example, the base member 112 may have one or more openings configured to receive the locking element 114 and/or fixation members 15 therethrough. By way of example only, the base member 112 may comprise a single or multi-hole button, plate, surgical nail (e.g., fibular nail), and the like. By way of example only, in the embodiment shown and described in FIGS. 18-20, the base member 112 comprises a round orthopedic button.

In some embodiments, the base member 112 includes a top or proximal surface 116, a bottom or distal surface 118, a central recess 119 formed within the top surface 116, an elongated central aperture 120 positioned within the central recess 119, and a pair of lateral apertures 121 positioned on either side of the central aperture 120. By way of example, the central aperture 120 is elongated and has a shape complementary to the shape of the elongated flange 132 of the locking element 114 to prevent the locking element from rotating when mated with the base member 112. The lateral apertures 121 are configured to enable passage of one or more fixation members 15 therethrough. In a preferred embodiment, each lateral aperture 121 is configured to receive a free end 74 of the fixation member 15 therethrough. In some embodiments, the button 112 further includes a sloped circumferential mating surface 122 surrounding the central recess 119 having a slope extending into the central recess 119 from the top surface 116. In some embodiments, the sloped mating surface 122 has a generally linear slope. In some embodiments, the sloped mating surface 122 may have a generally concave slope. In some embodiments, the sloped mating surface 122 may have a generally convex slope. In some embodiments, the sloped mating surface 122 may have a slope angle in the range of 1° to 90°. By way of example, the sloped mating surface 122 is complementary to the sloped mating surface 138 of the locking element 114 and cooperates with the sloped mating surface 138 of the locking element 114 to pinch the fixation element 15 therebetween under tension (e.g., at “pinch points” 123), to knotlessly lock the button assembly 110 during use.

In some embodiments, the locking element 114 has a head 130 and elongated flange 132 extending distally from the head 130. By way of example, the head 130 may have any shape, including but not limited to circular (as shown by way of example only in FIG. 19), square, oblong, oval, rectangular, triangular, etc. In some embodiments, the head 130 has a top or proximal side 134, a bottom or distal side 136, and a sloped mating surface 138 provided on the bottom side 136 extending around the periphery of the head 130. By way of example, the sloped mating surface 138 is complementary to the mating surface 122 of the button 112 and cooperates with the sloped mating surface 122 of the button 112 to pinch the fixation element 15 therebetween under tension (e.g., at “pinch points” 123), to knotlessly lock the button assembly 110 during use. In some embodiments, the sloped mating surface 138 has a generally linear slope. In some embodiments, the sloped mating surface 138 may have a generally convex slope. In some embodiments, the sloped mating surface 138 may have a generally concave slope. In some embodiments, the sloped mating surface 138 may have a slope angle in the range of 1° to 90°. In some embodiments, the head 130 may include a pair of concave recesses 139 configured for passage of the fixation member 15 therethrough. In some embodiments, the “pinch points”123 may be aligned with the concave recesses 139 such that each free end 74 of the fixation member 15 passes through a lateral aperture 121 of base member and is contacted by the locking element 114 within the concave recesses 139.

In some embodiments, the elongated flange 132 may have two or more transverse openings spaced vertically apart from one another. In some embodiments, the elongated flange 132 includes a first transverse opening 140 configured to allow passage of one or more loops of the fixation member 15 therethrough. In some embodiments, the elongated flange 132 may include a second transverse opening 142 configured to allow passage of a different one or more loops of the fixation member 15 therethrough. In some embodiments, passing only one loop of the fixation member 15 through each transverse opening may make tensioning of the fixation member 15 easier in that it reduces friction that may occur when multiple strands of the fixation member 15 pass through the same transverse opening. In some embodiments, the outer edges of the transverse openings 140 and/or 142 may be smooth, curved, and/or rounded to reduce friction on the fixation member 15 as it passes through the transverse openings. By way of example only, the elongated flange 132 may have rounded lateral sides 144. By way of example, the elongated flange 132 has a proximal end 146 and a distal end 148. The proximal end 146 is the portion of the elongated flange 132 that interfaces with and extends from the head 130 of the locking element 114. In some embodiments, the distal end 148 may comprise a smooth and/or rounded surface to minimize trauma to nearby patient bone or tissue.

In some embodiments, the elongated flange 132 may have a maximum width dimension w₁ having a value of not more than 2.5 mm. In some embodiments, the elongated flange 132 may have a maximum width dimension w₁ having a value between 1 mm and 2.5 mm. In some embodiments, the elongated flange 132 may have a length dimension l₁ measured from the proximal end 146 to the distal end 148. In some embodiments, the length dimension l₁ has a value between 1 mm and 30 mm. In some embodiments, the elongated flange 132 may have a length dimension l₁ having a value of 4 mm. In some embodiments, the elongated flange 132 (as well as the base member 112 and locking element 114) may be larger to fit specific anatomy.

By way of example, the knotless orthopedic fixation system 110 is configured for use in bone-to-bone and soft tissue-to-bone repair, by knotlessly securing the flexible fixation member 15 under tension. In some embodiments, the button 112 is engaged with a first bone segment, and the flexible fixation member 15 is secured to a second bone segment, soft tissue portion, or other member (e.g., plate, secondary button, etc.) and then passed through the button 112, to which it is locked under tension by way of the locking element 114. By way of example, when assembled with the button 112 and locking member 114, the flexible fixation member 15 comprises a distal looped portion 70 (e.g., which is the portion of the fixation member 15 that is attached to tissue, bone, or other member), one or more proximal looped portions 72 (e.g., which are passed through transverse openings 140, 142 of the elongated flange 132), and free ends 74 that extend proximally from the button 12 after passing through the “pinch points” 123.

By way of example, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 123 between the base member 112 and locking element 114 as described above, thereby maintaining the stabilization system 110 in a locked state.

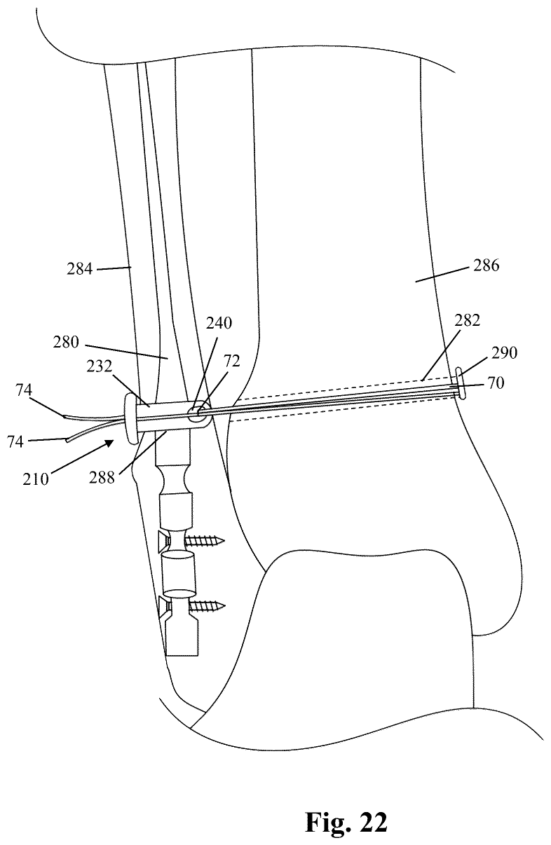

FIGS. 21-22 illustrate an example of a knotless orthopedic stabilization system 210 according to some embodiments of the present disclosure. By way of example, the knotless stabilization system 210 includes a base member (or “button”) 212, a locking element 214, and a flexible fixation member 15. In some embodiments, the base member 212 and locking element 214 cooperate to lock the fixation member 15 (e.g., surgical suture, surgical tape, tensioning member, etc.) therebetween after a desired tension has been applied to the fixation member. By way of example, the base member 212 may have any suitable shape, including but not limited to circular, oval, oblong, rectangular, etc. By way of example, the base member 112 may have one or more openings configured to receive the locking element 214 and/or fixation members 15 therethrough. By way of example only, the base member 212 may comprise a single or multi-hole button, plate, surgical nail (e.g., fibular nail), and the like. By way of example only, in the embodiment shown and described in FIGS. 21-22, the base member 112 comprises a round orthopedic button.

By way of example, the base member 212 is identical to base member 112 described above, and includes all features described in relation to base member 112 even if not specifically identified in this section. In some embodiments, the base member 212 includes a central recess 219 having an elongated central aperture 220 configured to receive the elongated flange 232 of the locking element 214 therethrough and a pair of lateral apertures 221 positioned on either side of the central aperture 220 and configured to enable passage of the fixation member 15 therethrough. In a preferred embodiment, each lateral aperture 221 is configured to receive one free end 74 of the fixation member 15 therethrough. In some embodiments, the button 212 further includes a sloped circumferential mating surface 222 surrounding the central recess 219 having a slope extending into the central recess 219. In some embodiments, the sloped mating surface 222 has a generally linear slope. In some embodiments, the sloped mating surface 222 may have a generally concave slope. In some embodiments, the sloped mating surface 222 may have a generally convex slope. In some embodiments, the sloped mating surface 222 may have a slope angle in the range of 1° to 90°. By way of example, the sloped mating surface 222 is complementary to the sloped mating surface 238 of the locking element 214 and cooperates with the sloped mating surface 238 of the locking element 214 to pinch the fixation element 15 therebetween under tension (e.g., at “pinch points” 223), to knotlessly lock the button assembly 210 during use.

By way of example, the locking element 214 is nearly identical to locking element 114 described above, and includes all features described in relation to base member 112 even if not specifically identified in this section, except that the locking element 214 has only one transverse aperture 240 as described below. In some embodiments, the locking element 214 has a head 230 and elongated flange 232 extending distally from the head 230. By way of example, the head 230 may have any shape, including but not limited to circular (as shown by way of example only in FIG. 21), square, oblong, oval, rectangular, triangular, etc. In some embodiments, the head 230 has a top side, a bottom side, and a sloped mating surface 238 provided on the bottom side extending around the periphery of the head 230. By way of example, the sloped mating surface 238 is complementary to the mating surface 222 of the button 212 and cooperates with the sloped mating surface 222 of the button 212 to pinch the fixation element 15 therebetween under tension (e.g., at “pinch points” 223), to knotlessly lock the button assembly 210 during use. In some embodiments, the sloped mating surface 238 has a generally linear slope. In some embodiments, the sloped mating surface 238 may have a generally convex slope. In some embodiments, the sloped mating surface 238 may have a generally concave slope. In some embodiments, the sloped mating surface 238 may have a slope angle in the range of 1° to 90°. In some embodiments, the head 230 may include a pair of concave recesses 239 configured for passage of the fixation member 15 therethrough. In some embodiments, the “pinch points” 223 may be aligned with the concave recesses 239 such that each free end 74 of the fixation member 15 passes through a lateral aperture 221 of base member and is contacted by the locking element 214 within the concave recesses 239.

By way of example, the elongated flange 232 has a proximal end 246 and a distal end 248. The proximal end 246 is the portion of the elongated flange 232 that interfaces with and extends from the head 230 of the locking element 214. In some embodiments, the distal end 248 may comprise a smooth and/or rounded surface to minimize trauma to nearby patient bone or tissue. In some embodiments, the elongated flange 232 may have a single transverse opening 240 positioned near the distal end 248 and configured to allow passage of one or more proximal loops 72 of the fixation member 15 therethrough. In some embodiments, the transverse opening has a width or diameter dimension. In some embodiments, the transverse opening 240 is spaced from the proximal end 246 a distance that is more than double the width dimension of the transverse opening 240. In some embodiments, the transverse opening 240 is spaced from the proximal end 246 a distance that is more than triple the width dimension of the transverse opening 240. In some embodiments, the outer edge of the transverse opening 240 may be smooth, curved, and/or rounded to reduce friction on the fixation member 15 as it passes through the transverse opening 240.

In some embodiments, the elongated flange 232 may have a maximum width dimension w₁ having a value of not more than 2.5 mm. In some embodiments, the elongated flange 232 may have a maximum width dimension w₁ having a value between 1 mm and 2.5 mm. In some embodiments, the elongated flange 232 may have a length dimension l₁ measured from the proximal end 246 to the distal end 248. In some embodiments, the length dimension l₁ has a value between 1 mm and 30 mm.

By way of example, the knotless orthopedic fixation system 210 is configured for use in bone-to-bone and soft tissue-to-bone repair, by knotlessly securing the flexible fixation member 15 under tension. In some embodiments, the button 212 is engaged with a first bone segment, and the flexible fixation member 15 is secured to a second bone segment, soft tissue portion, or other member (e.g., plate, secondary button, etc.) and then passed through the button 212, to which it is locked under tension by way of the locking element 214. By way of example, when assembled with the button 212 and locking member 214, the flexible fixation member 15 comprises a distal looped portion 70 (e.g., which is the portion of the fixation member 15 that is attached to tissue, bone, or other member), one or more proximal looped portions 72 (e.g., which are passed through transverse opening 240 of the elongated flange 232), and free ends 74 that extend proximally from the button 212 after passing through the “pinch points” 223.

By way of example, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 223 between the base member 212 and locking element 214 as described above, thereby maintaining the stabilization system 210 in a locked state.

As shown by way of example only in FIG. 22, in some embodiments, the knotless orthopedic stabilization system 210 may be used in an orthopedic fixation procedure with a plate, rod, nail, and the like as a supplemental stabilizing element rather than a primary fixation element. For example, as illustrated in FIG. 22, the system 210 may be used with a fibular nail 280 to stabilize the fixation procedure. For example, a transverse bone tunnel 282 may be drilled through a fibula 284 and also the tibia 286 at a location corresponding to a hole 288 in the fibular nail 280. A supplemental button or plate 290 is then shuttled through the bone tunnel 282 to the far side of the tibia 286 where it is positioned and secured with one or more fixation members 15 (e.g., by engaging with distal looped ends 70 of the fixation member 15). Proximal looped ends 72 of the fixation member 15 engage the transverse opening 240 of the elongated flange 232, which is positioned through the hole 288 in the fibular nail 200. In some embodiments, the elongated flange 232 may extend at least partially into the bone tunnel 282 in the fibula and/or tibia. The construct is then tensioned by pulling on the free ends 74 of the fixation member 15 as described above.

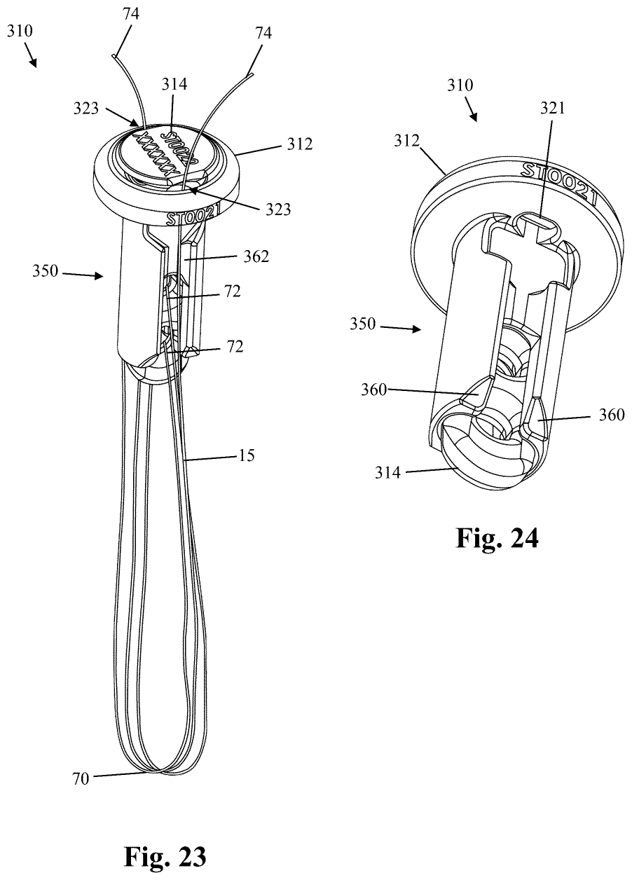

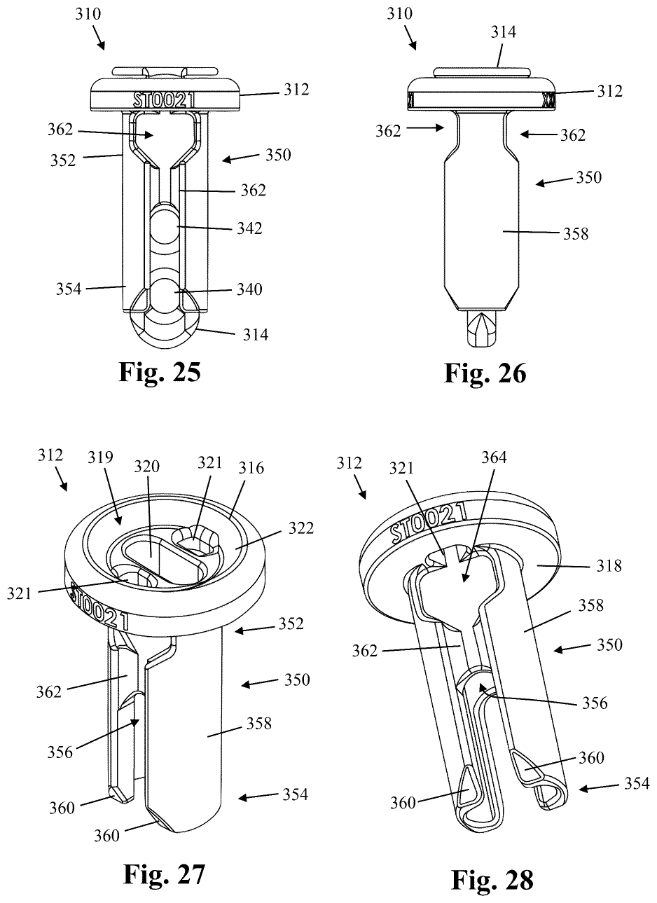

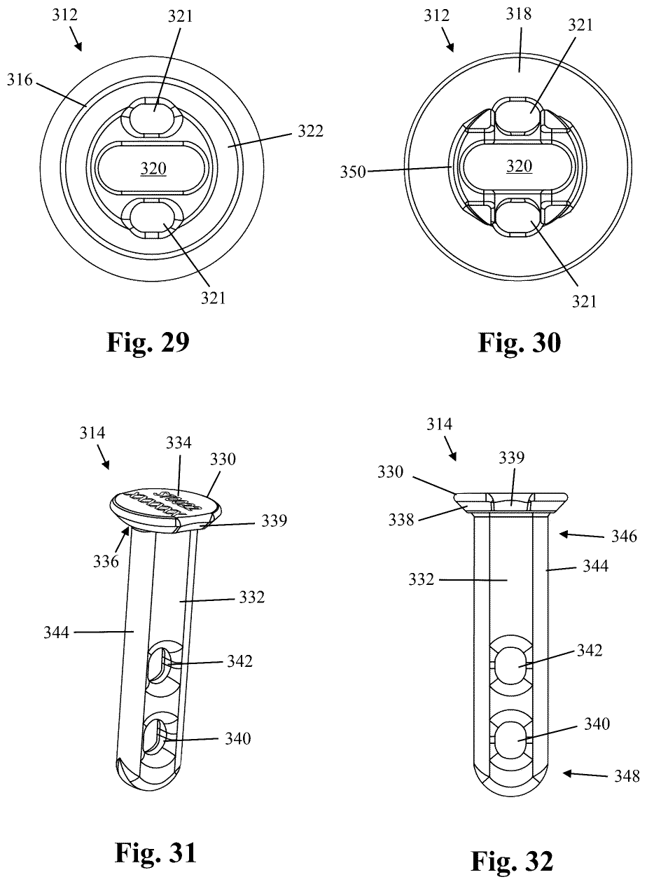

FIGS. 23-32 illustrate an example of a knotless orthopedic stabilization system 310 according to some embodiments of the present disclosure. By way of example, the knotless stabilization system 310 includes a base member (or “button”) 312, a locking element 314, and a flexible fixation member 15. In some embodiments, the base member 312 and locking element 314 cooperate to lock the fixation member 15 (e.g., surgical suture, surgical tape, tensioning member, etc.) therebetween after a desired tension has been applied to the fixation member. By way of example, the base member 312 may have any suitable shape, including but not limited to circular, oval, oblong, rectangular, etc. By way of example, the base member 312 may have one or more openings configured to receive the locking element 314 and/or fixation members 15 therethrough. By way of example only, the base member 312 may comprise a single or multi-hole button, plate, surgical nail (e.g., fibular nail), and the like. By way of example only, in the embodiment shown and described in FIGS. 23-32, the base member 312 comprises a round orthopedic button having a stabilizer member extending distally therefrom to provide stability of the fixation construct when used as a supplemental fixation element (e.g., with a plate, nail, etc.) or as a primary fixation element (e.g., when inserted into a bone tunnel).

In some embodiments, the base member 312 includes a top or proximal surface 316, a bottom or distal surface 318, a central recess 319 formed within the top surface 316, an elongated central aperture 320 positioned within the central recess 319, and a pair of lateral apertures 321 positioned on either side of the central aperture 320. By way of example, the central aperture 320 is elongated and has a shape complementary to the shape of the elongated flange 332 of the locking element 314 to prevent the locking element from rotating when mated with the base member 312. The lateral apertures 321 are configured to enable passage of one or more fixation members 15 therethrough. In a preferred embodiment, each lateral aperture 321 is configured to receive one free end 74 of the fixation member 15 therethrough. In some embodiments, the button 312 further includes a sloped circumferential mating surface 322 surrounding the central recess 319 having a slope extending into the central recess 319 from the top surface 316. In some embodiments, the sloped mating surface 322 has a generally linear slope. In some embodiments, the sloped mating surface 322 may have a generally concave slope. In some embodiments, the sloped mating surface 322 may have a generally convex slope. In some embodiments, the sloped mating surface 322 may have a slope angle in the range of 1° to 90°. By way of example, the sloped mating surface 322 is complementary to the sloped mating surface 138 of the locking element 314 and cooperates with the sloped mating surface 338 of the locking element 314 to pinch the fixation element 15 therebetween under tension (e.g., at “pinch points” 323), to knotlessly lock the button assembly 310 during use.

In some embodiments, the base member 312 includes a stabilizing member or jacket 350 extending distally from the bottom surface 318. By way of example, the stabilizing jacket 350 is configured for insertion or through into a bone channel or another fixation member (e.g., plate, nail, etc.) and stabilizes the elongated flange 332 of the locking element 314 to prevent the elongated flange 332 from moving during use (e.g., preventing back and forth rocking or “windshield wiper” motion) to ensure a more stable fixation construct. In some embodiments, the stabilizing jacket 350 comprises a proximal portion 352, a distal portion 354, and a central channel 356 extending longitudinally therethrough. In some embodiments, the central channel 356 is an extension of the central aperture 320 and is sized and configured to flushly receive the distal flange 332 therein so that the distal flange 332 cannot move in any direction other than a distal or proximal translation within the central channel 356. In some embodiments, the proximal portion 352 is configured to fully enclose the central channel 356. In some embodiments, the distal portion 354 does not fully enclose the central channel 356 so that the transverse openings 340, 342 are fully uncovered to enable unimpeded movement of the fixation members 15 therethrough. In some embodiments, the stabilizing jacket 350 further includes a smooth, curved outer surface 358 and a plurality of chamfered surfaces 360 at the distal end of the distal portion 354, both features which enable smooth insertion into a bone tunnel, for example. In some embodiments, the stabilizing jacket 350 further includes a pair of outer channels 362 configured to guide and protect the fixation member 15 as it exits the lateral apertures 321 of the base member 312. In some embodiments, the outer channels 362 do not extend proximally to the bottom surface 318 of the base member 312, creating an open space or void 364 that provides extra space if any bunching of the tension member 15 occurs during tensioning.

In some embodiments, the locking element 314 has a head 330 and elongated flange 332 extending distally from the head 330. By way of example, the head 330 may have any shape, including but not limited to circular (as shown by way of example only in FIG. 31), square, oblong, oval, rectangular, triangular, etc. In some embodiments, the head 330 has a top or proximal side 334, a bottom or distal side 336, and a sloped mating surface 338 provided on the bottom side 336 extending around the periphery of the head 330. By way of example, the sloped mating surface 338 is complementary to the mating surface 322 of the button 312 and cooperates with the sloped mating surface 322 of the button 312 to pinch the fixation element 15 therebetween under tension (e.g., at “pinch points” 323), to knotlessly lock the button assembly 310 during use. In some embodiments, the sloped mating surface 338 has a generally linear slope. In some embodiments, the sloped mating surface 338 may have a generally convex slope. In some embodiments, the sloped mating surface 338 may have a generally concave slope. In some embodiments, the sloped mating surface 338 may have a slope angle in the range of 1° to 90°. In some embodiments, the head 330 may include a pair of concave recesses 339 configured for passage of the fixation member 15 therethrough. In some embodiments, the “pinch points”323 may be aligned with the concave recesses 339 such that each free end 74 of the fixation member 15 passes through a lateral aperture 321 of base member 314 and is contacted by the locking element 314 within the concave recesses 339.

In some embodiments, the elongated flange 332 may have two or more transverse openings spaced vertically apart from one another. In some embodiments, the elongated flange 332 includes a first transverse opening 340 configured to allow passage of one or more loops 72 of the fixation member 15 therethrough. In some embodiments, the elongated flange 332 may include a second transverse opening 342 configured to allow passage of a different one or more loops 72 of the fixation member 15 therethrough. In some embodiments, passing only one loop of the fixation member 15 through each transverse opening may make tensioning of the fixation member 15 easier in that it reduces friction that may occur when multiple strands of the fixation member 15 pass through the same transverse opening. In some embodiments, the outer edges of the transverse openings 340 and/or 342 may be smooth, curved, and/or rounded to reduce friction on the fixation member 15 as it passes through the transverse openings. By way of example only, the elongated flange 332 may have rounded lateral sides 344. By way of example, the elongated flange 332 has a proximal end 346 and a distal end 348. The proximal end 346 is the portion of the elongated flange 332 that interfaces with and extends from the head 330 of the locking element 314. In some embodiments, the distal end 348 may comprise a smooth and/or rounded surface to minimize trauma to nearby patient bone or tissue.

In some embodiments, the elongated flange 332 may have a maximum width dimension w₁ having a value of not more than 2.5 mm. In some embodiments, the elongated flange 332 may have a maximum width dimension w₁ having a value between 1 mm and 2.5 mm. In some embodiments, the elongated flange 332 may have a length dimension l₁ measured from the proximal end 346 to the distal end 348. In some embodiments, the length dimension l₁ has a value between 1 mm and 30 mm. In some embodiments, the elongated flange 332 may have a length dimension l₁ having a value of 4 mm. In some embodiments, the elongated flange 332 is configured such that the distal end 348 extends beyond the distal end of the stabilizing jacket 350 so that the tension member 15 is not impeded by the stabilizing jacket 350 during use. In some embodiments, the elongated flange 332 (as well as the base member 312 and locking element 314) may be larger to fit specific anatomy. In some embodiments, the elongated flange 332 may have a maximum width dimension w₁ having a value of not more than 4.5 mm.

By way of example, the knotless orthopedic fixation system 310 is configured for use in bone-to-bone and soft tissue-to-bone repair, by knotlessly securing the flexible fixation member 15 under tension. In some embodiments, the button 312 is engaged with a first bone segment, and the flexible fixation member 15 is secured to a second bone segment, soft tissue portion, or other member (e.g., plate, secondary button, etc.) and then passed through the button 312, to which it is locked under tension by way of the locking element 314. By way of example, when assembled with the button 312 and locking member 314, the flexible fixation member 15 comprises a distal looped portion 70 (e.g., which is the portion of the fixation member 15 that is attached to tissue, bone, or other member), one or more proximal looped portions 72 (e.g., which are passed through transverse openings 340, 342 of the elongated flange 332), and free ends 74 that extend proximally from the button 312 after passing through the “pinch points” 323.

By way of example, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 323 between the base member 312 and locking element 314 as described above, thereby maintaining the stabilization system 310 in a locked state.

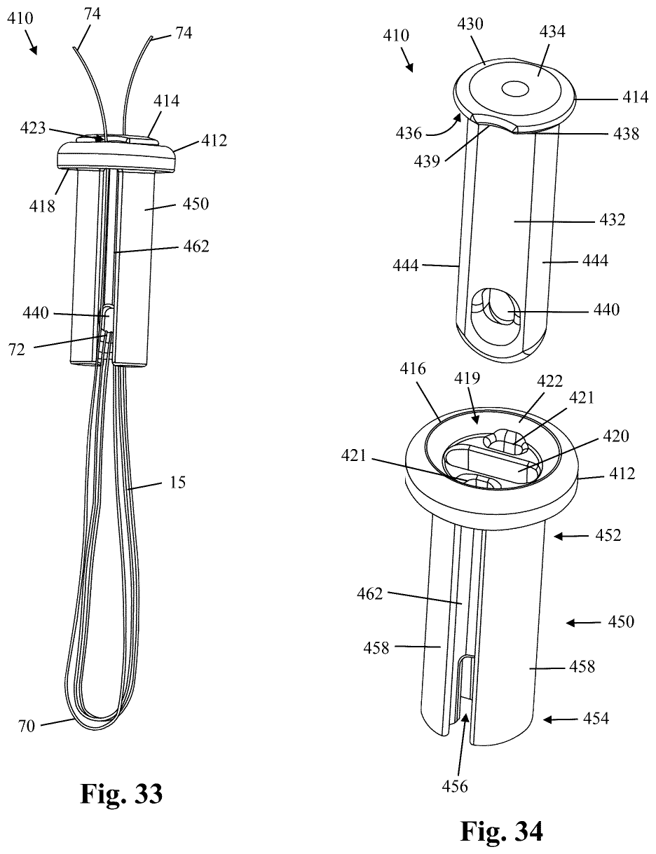

FIGS. 33-34 illustrate an example of a knotless orthopedic stabilization system 410 according to some embodiments of the present disclosure. By way of example, the knotless stabilization system 410 includes a base member (or “button”) 412, a locking element 414, and a flexible fixation member 15. In some embodiments, the base member 412 and locking element 414 cooperate to lock the fixation member 15 (e.g., surgical suture, surgical tape, tensioning member, etc.) therebetween after a desired tension has been applied to the fixation member. By way of example, the base member 412 may have any suitable shape, including but not limited to circular, oval, oblong, rectangular, etc. By way of example, the base member 412 may have one or more openings configured to receive the locking element 414 and/or fixation members 15 therethrough. By way of example only, the base member 412 may comprise a single or multi-hole button, plate, surgical nail (e.g., fibular nail), and the like. By way of example only, in the embodiment shown and described in FIGS. 33-34, the base member 412 comprises a round orthopedic button having a stabilizer member extending distally therefrom to provide stability of the fixation construct when used as a supplemental fixation element (e.g., with a plate, nail, etc.) or as a primary fixation element (e.g., when inserted into a bone tunnel).

In some embodiments, the base member 412 includes a top or proximal surface 416, a bottom or distal surface 418, a central recess 419 formed within the top surface 416, an elongated central aperture 420 positioned within the central recess 419, and a pair of lateral apertures 421 positioned on either side of the central aperture 420. By way of example, the central aperture 420 is elongated and has a shape complementary to the shape of the elongated flange 432 of the locking element 414 to prevent the locking element from rotating when mated with the base member 412. The lateral apertures 421 are configured to enable passage of one or more fixation members 15 therethrough. In a preferred embodiment, each lateral aperture 421 is configured to receive one free end 74 of the fixation member 15 therethrough. In some embodiments, the button 412 further includes a sloped circumferential mating surface 422 surrounding the central recess 419 having a slope extending into the central recess 419 from the top surface 416. In some embodiments, the sloped mating surface 422 has a generally linear slope. In some embodiments, the sloped mating surface 422 may have a generally concave slope. In some embodiments, the sloped mating surface 422 may have a generally convex slope. In some embodiments, the sloped mating surface 422 may have a slope angle in the range of 1° to 90°. By way of example, the sloped mating surface 422 is complementary to the sloped mating surface 438 of the locking element 414 and cooperates with the sloped mating surface 438 of the locking element 414 to pinch the fixation element 15 therebetween under tension (e.g., at “pinch points” 423), to knotlessly lock the button assembly 410 during use.

In some embodiments, the base member 412 includes a stabilizing member or jacket 450 extending distally from the bottom surface 418. By way of example, the stabilizing jacket 450 is configured for insertion or through into a bone channel or another fixation member (e.g., plate, nail, etc.) and stabilizes the elongated flange 432 of the locking element 414 to prevent the elongated flange 432 from moving during use (e.g., preventing back and forth rocking or “windshield wiper” motion) to ensure a more stable fixation construct. In some embodiments, the stabilizing jacket 450 comprises a proximal portion 452, a distal portion 454, and a central channel 456 extending longitudinally therethrough. In some embodiments, the central channel 456 is an extension of the central aperture 420 and is sized and configured to flushly receive the distal flange 432 therein so that the distal flange 432 cannot move in any direction other than a distal or proximal translation within the central channel 456. In some embodiments, the proximal portion 452 is configured to fully enclose the central channel 456. In some embodiments, the distal portion 454 does not fully enclose the central channel 456 so that the transverse opening 440 is fully uncovered to enable unimpeded movement of the fixation members 15 therethrough. In some embodiments, the stabilizing jacket 450 further includes a smooth, curved outer surface 458 which enables smooth insertion into a bone tunnel, for example. In some embodiments, the stabilizing jacket 450 further includes a pair of outer channels 462 configured to guide and protect the fixation member 15 as it exits the lateral apertures 421 of the base member 412. In some embodiments, the outer channels 462 extend proximally to the bottom surface 418 of the base member 412.

In some embodiments, the locking element 414 has a head 430 and elongated flange 432 extending distally from the head 430. By way of example, the head 430 may have any shape, including but not limited to circular (as shown by way of example only in FIG. 34), square, oblong, oval, rectangular, triangular, etc. In some embodiments, the head 430 has a top or proximal side 434, a bottom or distal side 436, and a sloped mating surface 438 provided on the bottom side 436 extending around the periphery of the head 430. By way of example, the sloped mating surface 438 is complementary to the mating surface 422 of the button 412 and cooperates with the sloped mating surface 422 of the button 412 to pinch the fixation element 15 therebetween under tension (e.g., at “pinch points” 423), to knotlessly lock the button assembly 410 during use. In some embodiments, the sloped mating surface 438 has a generally linear slope. In some embodiments, the sloped mating surface 438 may have a generally convex slope. In some embodiments, the sloped mating surface 438 may have a generally concave slope. In some embodiments, the sloped mating surface 438 may have a slope angle in the range of 1° to 90°. In some embodiments, the head 430 may include a pair of concave recesses 439 configured for passage of the fixation member 15 therethrough. In some embodiments, the “pinch points”423 may be aligned with the concave recesses 439 such that each free end 74 of the fixation member 15 passes through a lateral aperture 421 of base member 314 and is contacted by the locking element 414 within the concave recesses 439.

In some embodiments, the elongated flange 432 may have a single transverse opening 340 positioned at the distal end of the elongated flange 342 and configured to allow passage of one or more loops 72 of the fixation member 15 therethrough. In some embodiments, the outer edges of the transverse opening 440 may be smooth, curved, and/or rounded to reduce friction on the fixation member 15 as it passes through the transverse opening 440. By way of example only, the elongated flange 432 may have rounded lateral sides 444. By way of example, the elongated flange 432 has a proximal end 446 and a distal end 448. The proximal end 446 is the portion of the elongated flange 432 that interfaces with and extends from the head 430 of the locking element 414. In some embodiments, the distal end 448 may comprise a smooth and/or rounded surface to minimize trauma to nearby patient bone or tissue.

In some embodiments, the elongated flange 432 may have a maximum width dimension w₁ having a value of not more than 2.5 mm. In some embodiments, the elongated flange 432 may have a maximum width dimension w₁ having a value between 1 mm and 2.5 mm. In some embodiments, the elongated flange 432 may have a length dimension l₁ measured from the proximal end 446 to the distal end 448. In some embodiments, the length dimension l₁ has a value between 1 mm and 30 mm. In some embodiments, the elongated flange 432 may have a length dimension l₁ having a value of 4 mm. In some embodiments, the elongated flange 332 (as well as the base member 312 and locking element 314) may be larger to fit specific anatomy. In some embodiments, the elongated flange 332 may have a maximum width dimension w₁ having a value of not more than 4.5 mm.

By way of example, the knotless orthopedic fixation system 410 is configured for use in bone-to-bone and soft tissue-to-bone repair, by knotlessly securing the flexible fixation member 15 under tension. In some embodiments, the button 412 is engaged with a first bone segment, and the flexible fixation member 15 is secured to a second bone segment, soft tissue portion, or other member (e.g., plate, secondary button, etc.) and then passed through the button 412, to which it is locked under tension by way of the locking element 414. By way of example, when assembled with the button 412 and locking member 414, the flexible fixation member 15 comprises a distal looped portion 70 (e.g., which is the portion of the fixation member 15 that is attached to tissue, bone, or other member), one or more proximal looped portions 72 (e.g., which are passed through transverse opening 440 of the elongated flange 432), and free ends 74 that extend proximally from the button 412 after passing through the “pinch points” 423.

By way of example, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 423 between the base member 412 and locking element 414 as described above, thereby maintaining the stabilization system 410 in a locked state.

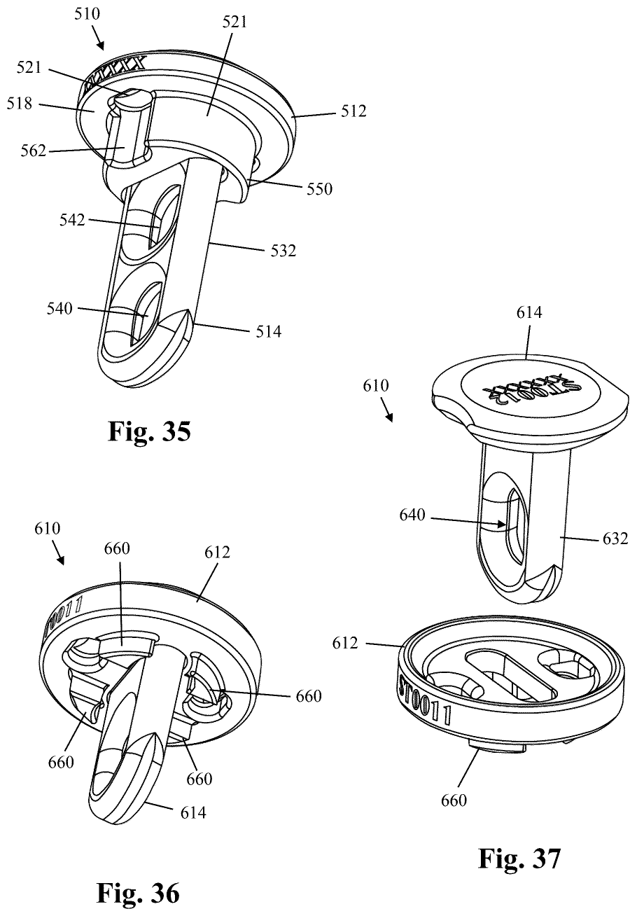

FIG. 35 illustrate an example of a knotless orthopedic stabilization system 510 according to some embodiments of the present disclosure. By way of example, the knotless stabilization system 510 includes a base member (or “button”) 512, a locking element 514, and a flexible fixation member (not shown, but same as flexible fixation member 15 described above). In some embodiments, the base member 512 and locking element 514 cooperate to lock a fixation member (e.g., surgical suture, surgical tape, tensioning member, etc.) therebetween after a desired tension has been applied to the fixation member. By way of example, the base member 512 may have any suitable shape, including but not limited to circular, oval, oblong, rectangular, etc. By way of example, the base member 512 may have one or more openings configured to receive the locking element 514 and/or fixation members therethrough. By way of example only, the base member 512 may comprise a single or multi-hole button, plate, surgical nail (e.g., fibular nail), and the like. By way of example only, in the embodiment shown and described in FIG. 35, the base member 512 comprises a round orthopedic button having a stabilizer member extending distally therefrom to provide stability of the fixation construct when used as a supplemental fixation element (e.g., with a plate, nail, etc.) or as a primary fixation element (e.g., when inserted into a bone tunnel).

By way of example only, the base member 512 is identical to the base member 412 described above, and any feature described in regard to base member 412 is applicable to base member 512 as well, save for the stabilizing jacket 550, which will be described in detail herein. By way of example only, the locking element 514 is identical to locking element 114 described above, and all features described in regard to locking element 112 above also apply to locking element 512 of the present example embodiment.

In some embodiments, the base member 512 includes a stabilizing member or jacket 550 extending distally from the bottom surface 518. By way of example, the stabilizing jacket 550 is configured for insertion or through into a bone channel or another fixation member (e.g., plate, nail, etc.) and stabilizes the elongated flange 532 of the locking element 514 to prevent the elongated flange 532 from moving during use (e.g., preventing back and forth rocking or “windshield wiper” motion) to ensure a more stable fixation construct. In some embodiments, the stabilizing jacket 550 is configured to cover only a proximal portion of the elongated flange 532 of the locking element 514, and includes a central channel 556 extending longitudinally therethrough. In some embodiments, the central channel 556 is an extension of the central aperture of the button 512 and is sized and configured to flushly receive the distal flange 532 therein so that the distal flange 532 cannot move in any direction other than a distal or proximal translation within the central channel 556. In some embodiments, central channel 556 is fully enclosed, but does not extend past the transverse openings 540, 542 of the distal flange 532 so that the transverse openings 540, 542 are fully uncovered to enable unimpeded movement of the fixation members 15 therethrough. In some embodiments, the stabilizing jacket 550 further includes a smooth, curved outer surface 558 which enables smooth insertion into a bone tunnel, for example. In some embodiments, the stabilizing jacket 550 further includes a pair of outer channels 562 configured to guide and protect the fixation member 15 as it exits the lateral apertures 521 of the base member 512. In some embodiments, the outer channels 562 extend proximally to the bottom surface 518 of the base member 512.

By way of example, the knotless orthopedic stabilization system 510 is configured for use in bone-to-bone and soft tissue-to-bone repair, by knotlessly securing the flexible fixation member under tension. Use and operation of the stabilization system 510 of the present embodiment mirrors the other embodiments described above.

FIGS. 36-37 illustrate an example of a knotless orthopedic stabilization system 610 that has been modified for use with a fixation plate, for example. In this example embodiments, the system 610 includes a base member 612 and a locking element 614 and is configured for use with a flexible fixation member in the manner described consistently throughout this disclosure. The locking element 614 is identical to the locking element 114 described above except that the locking element of the present embodiment (as shown by way of example only in FIG. 36) has only one transverse opening 640, however the locking element 614 may have any number of transverse openings. Notably, the bottom side 618 of the base member 612 includes a plurality of boss members 660 that are configured for insertion into an existing fixation aperture or screw hole of a fixation plate, for example, so that the system 610 may then be used with the plate in the manner described consistently herein with other embodiments. In some embodiments, the height of the boss members 660 can be varied to alter the orientation of the locking element relative to the plate, for example to change the angle of the elongated flange 632 as it extends through the plate, which may enable fixation with the flexible fixation member at a more extreme angle.

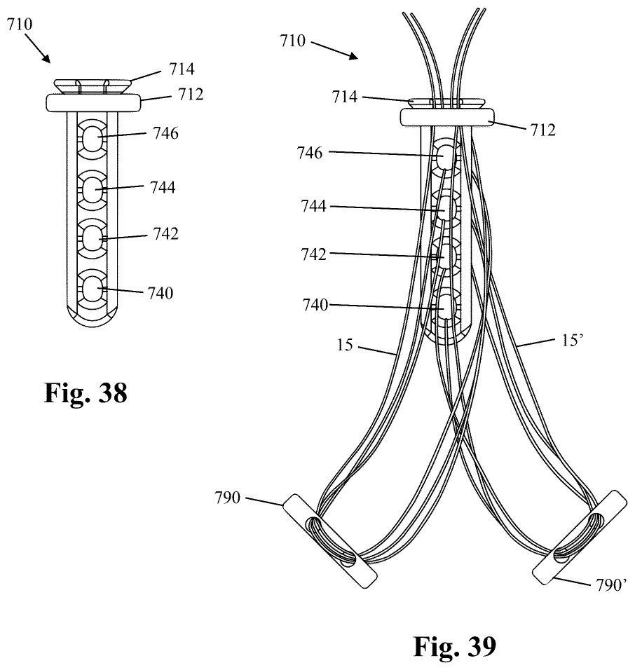

FIGS. 38-39 illustrate an example of a knotless orthopedic stabilization system 710 according to some embodiments. By way of example, the stabilization system 710 includes a base member or button 712, a locking element 714, and a pair of fixation members 15, 15′. The stabilization system 710 is similar to stabilization system 110 described above, with the difference being the locking element 714 includes an elongated flange 732 having four transverse apertures 740, 742, 744, 746. By way of example, the locking element 714 enables multiple fixation members 15, 15′ to be used and still have a single strand per transverse aperture. In some embodiments, the fixation members 15, 15′ may be attached at a distal looped end to a tissue, bone, or other member. For example, as shown in FIG. 39, each fixation member 15, 15′ is connected to a secondary button 790, 790′, respectively.

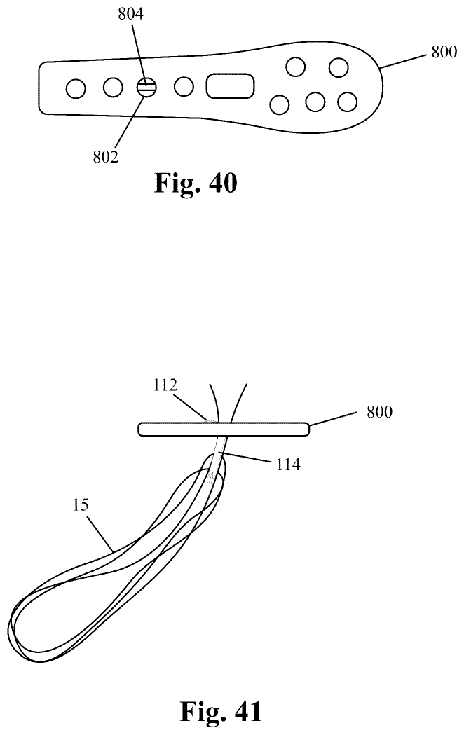

In some embodiments, the knotless fixation assembly disclosed herein may also be locked into a fixation plate in a certain direction to allow for maximum structural support by dialing or biasing the passage of the fixation members at an angle that may be performed by the operator of the device. In some embodiments, the locking element may be pivotable so that the flange may extend at different or variable angles through the button and then be locked in place. In some embodiments, the locking element and/or button and/or fixation plate may be modified to allow for various locking angles. As illustrated in FIGS. 40-41, in some embodiments, a fixation plate 800 or button may include an aperture 802 configured to receive the button therein having an angled slot 804 configured to receive the flange 132 of the locking element 114 threrethrough. By way of example, the slot 804 may be angled or directionally oriented in the direction that the user wants the flange 132 to extend. In some embodiments, the same feature can be achieved by way of an extender on the button (for example).

In any embodiment described herein, tensioning of the fixation members 15 is performed by pulling the free ends 74 of the fixation members 15 which will reduce the distance between the proximal looped end(s) 72 and the distal looped end(s) 70, and is completed when appropriate tension is achieved between two firm end points that increase the tension in the fixation member 15 which allows for pinching of the fixation members 15 at pinch points 23 between the base member 12 (and variants) and locking element 14 (and variants) as described herein, thereby maintaining the stabilization system 10 in a locked state.

Other embodiments may include single or multiple openings of the locking element, which by way of example only may be 4 mm or more in length. For example, in some embodiments, the flange portion of the locking element may be between 1 mm and 30 mm in length. In some embodiments, the button may have one or more openings. In some embodiments, the button may have extensions that extend into bone and provide additional stability to the bone, device, or other implants used. In some embodiments, the locking assembly has more than one mating surface and can be used for fixation members for soft tissue, and/or bone to bone, and/or the loops may be connected to another device on the far cortex (for example).

In some embodiments, the locking button assembly with fixation members that connect to one or multiple fixation members that allow for tensioning a plurality of constructs in conjunction with one or more knotless assemblies.

In some embodiments, the button and/or locking element can be made from titanium, stainless steel, peek, polymers, 3D-printing, or similar materials used in the industry to manufacture the button device. The manufacturing process can be machining, molding, 3D-printing, or other similar acceptable processes used in the industry. The fixation members can be suture, sutures, nitinol, other materials that can be used to fix soft tissue to bone fixation members may be braided, non-braided, can have a core or coreless.

In some embodiments, the design dimensions and the shape varies on the design anatomy, preferred technique, material and process use or additional devices that are to be implanted with this knotless system. The embodiments are examples of design that can be modified to suit the patient's needs.

We claim:

1. An orthopedic stabilization system comprising:
a base member having a proximal side, a distal side, a central recess formed in the proximal side, and a central opening positioned within the central recess and extending therethrough from the proximal side to the distal side;
a locking element having a proximal side, a distal side, and an elongated flange having an oblong cross-sectional shape extending distally from the distal side of the locking element, the elongated flange having at least two transverse openings spaced longitudinally apart from one another, the locking element configured to mate with the base member such that the elongated flange extends through the central opening and the at least two transverse openings are positioned distal of the distal side of the base member; and
a flexible fixation member configured to form into a looped orientation having two free ends, a distal loop portion, and a plurality of proximal loop portions, the flexible fixation member configured to pass through the central opening in the base member and between the base member and the locking element such that the two free ends are disposed outwardly in a proximal direction from the proximal side of the base member;
wherein a first of the plurality of proximal loop portions is configured to pass through a first of the at least two transverse openings in the elongated flange, a second of the plurality of proximal loop portions is configured to pass through a second of the at least two transverse openings in the elongated flange, and the distal loop portion is configured to engage a tissue, bone, or other member.

2. The orthopedic stabilization system of claim 1, wherein the central opening has a perimeter and further comprises a first pair of opposing shaped perimeter recesses formed within said perimeter, each of the first pair of opposing shaped perimeter recesses configured to flushly engage a portion of the elongated flange.

3. The orthopedic stabilization device of claim 2, wherein the central opening further comprises a second pair of opposing shaped perimeter recesses formed within said perimeter and different from the first pair of opposing shaped perimeter recesses, the second pair of opposing shaped perimeter recesses configured to enable passage of the flexible fixation member through the base member.

4. The orthopedic stabilization device of claim 3, wherein the first pair of opposing perimeter recesses has a first size dimension, the second pair of opposing perimeter recesses has a second size dimension, and the first size dimension is greater than the second size dimension.

5. The orthopedic stabilization device of claim 2, wherein the first pair of opposing perimeter recesses and the second pair of opposing perimeter recesses are distributed about the perimeter of the central opening at 90° intervals from one another.

6. The orthopedic stabilization device of claim 2, wherein the base member has a longitudinal axis extending therethrough, and the first pair of opposing shaped perimeter recesses are positioned about the perimeter of the central opening at an oblique angle relative to the longitudinal axis.

7. The orthopedic stabilization device of claim 6, wherein the oblique angle is 45°.

8. The orthopedic stabilization device of claim 1, wherein the locking element further comprises one or more retention members positioned on the elongated flange and configured to engage the base member to prevent dislodging of the locking element from the base member when the locking element is coupled to the base member and the retention members are positioned distal of the base member.

9. The orthopedic stabilization device of claim 1, wherein the retention members are configured to deform as the elongated flange is inserted through the central opening during coupling of the base member and locking element.

10. The orthopedic stabilization device of claim 9, wherein the retention members are configured to return to their normal configuration when the retention members are distally clear of the central opening during coupling of the base member and locking element.

11. The orthopedic stabilization device of claim 10, wherein the base member has one or more undercut recesses formed in the distal side and configured to receive the retention members therein, when the retention members are distally clear of the central opening during coupling of the base member and locking element.

12. The orthopedic stabilization device of claim 1, wherein central recess has a first peripheral mating surface.

13. The orthopedic stabilization device of claim 12, wherein the distal side of the locking element as a second peripheral mating surface.

14. The orthopedic stabilization device of claim 13, the flexible fixation member is configured to pass between the first and second peripheral mating surfaces.

15. The orthopedic stabilization device of claim 13, wherein the first and second mating surfaces are configured to cooperate to capture the flexible fixation members with one or more pinch points that maintain the flexible fixation members under tension in a locked state.
